(19) United States
(12) Patent Application Publication
(10) Pub. No.: US 2006/0052345 A1
(43) Pub. Date: Mar. 9, 2006

(54) Quinazolinone compounds as calcilytics

(75) Inventors: Irina Shcherbakova, Midvale, UT (US); Manuel Balandrin, Sandy, UT (US); John Fox, Salt Lake City, UT (US); William L Heaton, Salt Lake City, UT (US); Rebecca L Conklin, Park City, UT (US); Damon I Papac, Salt Lake City, UT (US)

Correspondence Address: Kevin B Laurence;Stoel Rives, One Utah Center, 201 South Main Street Suite 1100, Salt Lake City, UT, 84111, US

(73) Assignee: NPS Pharmaceuticals, Inc., Salt Lake City, UT (US)

(21) Appl. No.: 10/531,161

(22) PCT Filed: Nov. 4, 2003
(86) PCT No.: PCT/US03/35162

§ 371 Date: Apr. 12, 2005

Related U.S. Application Data
(60) Provisional application No. 60/423,663, filed on Nov. 4, 2002.

(51) Int. Cl. A61K 31/675; A61K 31/519; A61K 31/517; C07D 487/02; C07D 403/02; C07D 239/91

(52) U.S. Cl. 514/81; 514/264.1; 514/266.31; 514/266.21; 544/244; 544/284; 544/286; 544/279

(57) ABSTRACT

Various calcilytic compounds and pharmaceutical compositions containing these compounds are disclosed. Calcilytic compounds are compounds capable of inhibiting calcium receptor activity. Techniques which can be used to obtain calcilytic compounds and uses of calcilyitc compounds as calcium receptor antagonists are also disclosed.

### FIELD OF INVENTION

The present invention relates to substituted 3H-quinazolin-4-ones able to inhibit calcium receptor activity and the use of such compounds. Preferably, the compounds described herein are administered to patients to achieve a therapeutic effect.

### BACKGROUND OF THE INVENTION

The present invention relates to novel calcilytic compounds, pharmaceutical compositions containing these compounds and their use as calcium receptor antagonists.

In mammals, extracellular Ca²⁺ is under rigid homeostatic control and regulates various processes such as blood clotting, nerve and muscle excitability, and proper bone formation. Extracellular Ca²⁺ inhibits the secretion of parathyroid hormone (“PTH”) from parathyroid cells, inhibits bone resorption by osteoclasts, and stimulates secretion of calcitonin from C-cells. Calcium receptor proteins enable certain specialized cells to respond to changes in extracellular Ca²⁺ concentration.

PTH is the principal endocrine factor regulating Ca²⁺ homeostasis in the blood and extracellular fluids. PTH, by acting on bone and kidney cells, increases the level of Ca²⁺ in the blood. This increase in extracellular Ca²⁺ then acts as a negative feedback signal, depressing PTH secretion. The reciprocal relationship between extracellular Ca²⁺ and PTH secretion forms an important mechanism maintaining bodily Ca²⁺ homeostasis.

Extracellular Ca²⁺ acts directly on parathyroid cells to regulate PTH secretion. The existence of a parathyroid cell surface protein which detects changes in extracellular Ca²⁺ has been confirmed. See Brown et al., Nature 366:574, 1993. In parathyroid cells, this protein, the calcium receptor, acts as a receptor for extracellular Ca²⁺, detects changes in the ion concentration of extracellular Ca²⁺, and initiates a functional cellular response, PTH secretion.

Extracellular Ca²⁺ influences various cell functions, reviewed in Nemeth et al., Cell Calcium 11:319, 1990. For example, extracellular Ca²⁺ plays a role in parafollicular (C-cells) and parathyroid cells. See Nemeth, Cell Calcium 11:323, 1990. The role of extracellular Ca²⁺ on bone osteoclasts has also been studied. See Zaidi, Bioscience Reports 10:493, 1990.

Various compounds are known to mimic the effects of extra-cellular Ca²⁺ on a calcium receptor molecule. Calcilytics are compounds able to inhibit calcium receptor activity, thereby causing a decrease in one or more calcium receptor activities evoked by extracellular Ca²⁺. Calcilytics are useful as lead molecules in the discovery, development, design, modification and/or construction of useful calcium modulators, which are active at Ca²⁺ receptors. Such calcilytics are useful in the treatment of various disease states characterized by abnormal levels of one or more components, e.g., polypeptides such as hormones, enzymes or growth factors, the expression and/or secretion of which is regulated or affected by activity at one or more Ca²⁺ receptors. Target diseases or disorders for calcilytic compounds include diseases involving abnormal bone and mineral homeostasis.

Abnormal calcium homeostasis is characterized by one or more of the following activities: an abnormal increase or decrease in serum calcium; an abnormal increase or decrease in urinary excretion of calcium; an abnormal increase or decrease in bone calcium levels (for example, as assessed by bone mineral density measurements); an abnormal absorption of dietary calcium; an abnormal increase or decrease in the production and/or release of messengers which affect serum calcium levels such as PTH and calcitonin; and an abnormal change in the response elicited by messengers which affect serum calcium levels.

Thus, calcium receptor antagonists offer a unique approach towards the pharmacotherapy of diseases associated with abnormal bone or mineral homeostasis, such as hypoparathyroidism, osteosarcoma, periodontal disease, fracture healing, osteoarthritis, rheumatoid arthritis, Paget's disease, humoral hypercalcemia associated with malignancy and fracture healing, and osteoporosis.

### SUMMARY OF THE INVENTION

The present invention features calcilytic compounds. “Calcilytic compounds” refer to compounds able to inhibit calcium receptor activity. The ability of a compound to “inhibit calcium receptor activity” means that the compound causes a decrease in one or more calcium receptor activities evoked by extracellular Ca²⁺.

The use of calcilytic compounds to inhibit calcium receptor activity and/or achieve a beneficial effect in a patient are described below. Also described below are techniques which can be used to obtain additional calcilytic compounds.

An example of featured calcilytic compounds are Structure I, representing 2,3,5,6,7,8-substituted 3H-quinazolin-4-ones having the chemical formula: 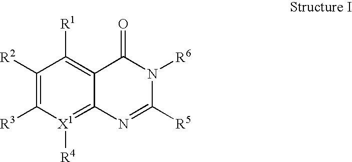
wherein:

R¹, R², and R³ is each independently selected from one of: H, halogen, CN, CF₃, OCF₃, lower alkyl, lower alkoxy, NH-acetyl, NH-lower alkyl, NH-alkylaryl, N(lower alkyl)₂, C(O)OH, C(O)O-lower alkyl, C(O)NH-lower alkyl, C(O)N(lower alkyl)₂, OH, OC(O)-lower alkyl, OC(O)-lower alkylamino, OC(O)-lower alkyl-N(lower alk)₂, OP(O)(OH)₂;
R⁴ is optional and is selected from one of: H, halogen, CN, CF₃, OCF₃, lower alkyl, lower alkoxy, NH-acetyl, NH-lower alkyl, NH-alkylaryl, N(lower alkyl)₂, C(O)OH, C(O)O-lower alkyl, C(O)NH-lower alkyl, C(O)N(lower alkyl)₂, OH, OC(O)-lower alkyl, OC(O)-lower alkylamino, OC(O)-lower alkyl-N(lower alk)₂, OP(O)(OH)₂;
X¹ is selected from one of C and N;
R⁵ is selected from one of: H, lower alkyl, or aryl or alkaryl group, which may have 1 to 3 substituents in the aryl ring each selected from one of: H, halogen, CN, CF₃, OCF₃, lower alkyl, NH-alkylaryl, N(lower alkyl)₂, lower alkoxy, OH, OC(O)-lower alkyl, OC(O)-lower alkylamino, OC(O)-lower alkyl-N(lower alkyl)₂, OP(O)(OH)₂; and
R⁶ is selected from one of: H, lower alkyl, or a group of the formula —(CH₂)ₙ—X²—R⁷ wherein n is 0, 1, or 2, X² is lower alk, O, C(O), CH(OH) or a single bond, R⁷ is an aryl group which may have 1 to 3 substituents on the aryl ring each selected from the group consisting of: H, halogen, CN, CF₃, OCF₃, lower alkyl, lower alkoxy, NH-lower alkyl NH-alkylaryl, N(lower alkyl)₂, OH, OC(O)-lower alk, OC(O)-lower alkylamino, OC(O)-lower alkyl-N(lower alk)₂, OP(O)(OH)₂,
and pharmaceutically acceptable salts and complexes thereof.

“Independently selected,” with reference to functional groups (such as R¹, R² and R³) means that the functional groups may be selected to be different or the same as each other.

“Alk” refers to either alkyl or alkenyl. “Lower alk” refers to either lower alkyl or lower alkenyl, preferably lower alkyl.

“Alkenyl” refers to an optionally substituted hydrocarbon group containing at least one carbon-carbon double bond between the carbon atoms and containing 2-6 carbon atoms joined together. The alkenyl hydrocarbon group may be straight-chain. Straight-chain alkenyl preferably has 2 to 4 carbons.

“Alkyl” refers to an optionally substituted hydrocarbon group joined by single carbon-carbon bonds and having 1 to 6 carbon atoms joined together. The alkyl hydrocarbon group may be straight-chain or contain one or more branches. Branched- and straight-chain alkyl preferably have 1 to 4 carbons, each of which may be optionally substituted. Alkyl substituents are each independently selected from the group consisting of: lower alkyl, unsubstituted aryl, OH, NH₂, NH-lower alkyl, and N(lower alkyl)₂. Preferably, no more than two substituents are present. Even more preferably, alkyl is a lower alkyl which is unsubstituted branched- or straight-chain alkyl having 2 to 4 carbons.

“Aryl” refers to an optionally substituted aromatic group with at least one ring having a conjugated or fused ring systems. Aryl includes carbocyclic aryl, heterocyclic aryl and biaryl groups, all of which may be optionally substituted. Preferably, the aryl is either optionally substituted phenyl or optionally substituted pyridyl.

“Alkoxy” refers to oxygen joined to an unsubstituted alkyl 1 to 4 carbon atoms in length, preferably 1 to 2 carbons in length. More preferably, the alkoxy is methoxy.

Preferred compounds useful in the present invention are selected from the group consisting of:
2-(2-hydroxy-phenyl)-3-phenethyl-3H-quinazolin-4-one;
2-(2,5-dihydroxy-phenyl)-3-phenethyl-3H-quinazolin-4-one;
2-(3-hydroxy-phenyl)-3-phenethyl-3H-quinazolin-4-one;
2-(2-hydroxy-phenyl)-3-(2-phenoxy-ethyl)-3H-quinazolin-4-one;
3-[2-(4-fluoro-phenyl)-ethyl]-2-(2-hydroxy-phenyl)-3H-quinazolin-4-one;
3-[2-(3-fluoro-phenyl)-ethyl]-2-(2-hydroxy-phenyl)-3H-quinazolin-4-one;
3-[2-(2-fluoro-phenyl)-ethyl]-2-(2-hydroxy-phenyl)-3H-quinazolin-4-one;
3-[2-(3-chloro-phenyl)-ethyl]-2-(2-hydroxy-phenyl)-3H-quinazolin-4-one;
3-[2-(2-chloro-phenyl)-ethyl]-2-(2-hydroxy-phenyl)-3H-quinazolin-4-one;
2-(2-hydroxy-phenyl)-3-[2-(4-methoxy-phenyl)-ethyl]-3H-quinazolin-4-one;
2-(2-hydroxy-phenyl)-3-(2-p-tolyl-ethyl)-3H-quinazolin-4-one; 2-(2-hydroxy-phenyl)-6-methyl-3-phenethyl-3H-quinazolin-4-one;
6-fluoro-2-(2-hydroxy-phenyl)-3-phenethyl-3H-quinazolin-4-one;
6-chloro-2-(2-hydroxy-phenyl)-3-phenethyl-3H-quinazolin-4-one;
2-(2-hydroxy-phenyl)-3-phenethyl-5-phenethylamino-3H-quinazolin-4-one;
2-(2-hydroxy-phenyl)-5-methyl-3-phenethyl-3H-quinazolin-4-one;
7-chloro-2-(2-hydroxy-phenyl)-3-phenethyl-3H-quinazolin-4-one;
2-(2-hydroxy-phenyl)-8-methyl-3-phenethyl-3H-quinazolin-4-one;
6-fluoro-3-[2-(3-fluoro-phenyl)-ethyl]-2-(2-hydroxy-phenyl)-3H-quinazolin-4-one;
6-fluoro-3-[2-(2-fluoro-phenyl)-ethyl]-2-(2-hydroxy-phenyl)-3H-quinazolin-4-one;
7-fluoro-3-[2-(3-fluoro-phenyl)-ethyl]-2-(2-hydroxy-phenyl)-3H-quinazolin-4-one;
3-[2-(2-fluoro-phenyl)-ethyl]-2-(2-hydroxy-phenyl)-5-methyl-3H-quinazolin-4-one;
3-[2-(3-fluoro-phenyl)-ethyl]-2-(2-hydroxy-phenyl)-5-methyl-3H-quinazolin-4-one;
3-[2-(3-fluoro-phenyl)-ethyl]-2-(2-hydroxy-phenyl)-6-methyl-3H-quinazolin-4-one;
3-[2-(2-fluoro-phenyl)-ethyl]-2-(2-hydroxy-phenyl)-6-methyl-3H-quinazolin-4-one;
6-chloro-3-[2-(3-fluoro-phenyl)-ethyl]-2-(2-hydroxy-phenyl)-3H-quinazolin-4-one;
6-chloro-3-[2-(2-fluoro-phenyl)-ethyl]-2-(2-hydroxy-phenyl)-3H-quinazolin-4-one;
3-[2-(3-fluoro-phenyl)-ethyl]-2-(2-hydroxy-phenyl)-6-methoxy-3H-quinazolin-4-one;
3-[2-(3-fluoro-phenyl)-ethyl]-6-hydroxy-2-(2-hydroxy-phenyl)-3H-quinazolin-4-one; acetic acid 2-{6-fluoro-3-[2-(3-fluoro-phenyl)-ethyl]-4-oxo-3,4-dihydro-quinazolin-2-yl}-phenyl ester;
3-[2-(3-fluoro-phenyl)-ethyl]-2-(2-hydroxy-phenyl)-8-methoxy-3H-quinazolin-4-one, isobutyric acid 2-{6-fluoro-3-[2-(3-fluoro-phenyl)-ethyl]-4-oxo-3,4-dihydro-quinazolin-2-yl}-phenyl ester;
sodium salt of 6-fluoro-3-[2-(3-fluoro-phenyl)-ethyl]-2-(2-hydroxy-phenyl)-3H-quinazolin-4-one;
8-chloro-2-(2-hydroxy-phenyl)-3-phenethyl-3H-quinazolin-4-one;
7-chloro-3-[2-(3-fluoro-phenyl)-ethyl]-2-(2-hydroxy-phenyl)-3H-quinazolin-4-one;
7-chloro-3-[2-(2-fluoro-phenyl)-ethyl]-2-(2-hydroxy-phenyl)-3H-quinazolin-4-one;
2-(2-hydroxy-phenyl)-3-(2-pyridin-3-yl-ethyl)-3H-quinazolin-4-one;
6-fluoro-2-(2-hydroxy-phenyl)-3-(2-pyridin-3-yl-ethyl)-3H-quinazolin-4-one;
2-(2-hydroxy-phenyl)-3-phenethyl-3H-pyrido[2,3-d]pyrimidin-4-one;
3-[2-(3-fluoro-phenyl)-ethyl]-2-(2-hydroxy-phenyl)-3H-pyrido[2,3-d]pyrimidin-4-one;
3-(1,1-dimethyl-3-phenyl-propyl)-6-fluoro-2-(2-hydroxy-phenyl)-3H-quinazolin-4-one;
methylamino-acetic acid 2-{6-fluoro-3-[2-(3-fluoro-phenyl)-ethyl]-4-oxo-3,4-dihydro-quinazolin-2-yl}-phenyl ester hydrochloride;
6-fluoro-2-(2-hydroxy-phenyl)-3-(2-phenyl-propyl)-3H-quinazolin-4-one;
6-fluoro-2-(2-hydroxy-phenyl)-3-(R-2-phenyl-propyl)-3H-quinazolin-4-one;
6-fluoro-2-(2-hydroxy-phenyl)-3-(S-2-phenyl-propyl)-3H-quinazolin-4-one;
6-fluoro-2-(2-hydroxy-phenyl)-3-(3-phenyl-propyl)-3H-quinazolin-4-one.

### BRIEF DESCRIPTION OF THE DRAWINGS

FIG. 1 is a chart showing plasma levels of compound 17 from 0 to 30 minutes following intravenous injection in normal rats.

FIG. 2 is a chart showing plasma PTH levels from 30 minutes before to 30 minutes following intravenous injection of compound 17 in normal rats.

### DETAILED DESCRIPTION OF THE INVENTION

The present application demonstrates the ability of calcilytic compounds to exert a physiologically relevant effect on a cell by illustrating the ability of such compounds to increase PTH secretion and also identifies a target site for calcilytic compounds.

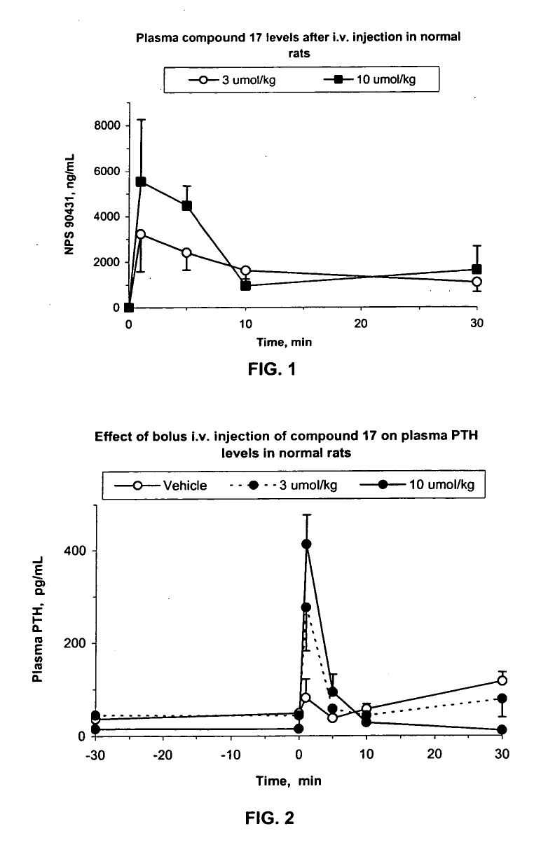

2-(2-Hydroxy-phenyl)-3-phenethyl-3H-quinazolin-4-one (compound 17) (3 or 10 μmol/kg) or vehicle was administered by intravenous injection over about 15 seconds to normal conscious male Sprague-Dawley rats with chronic indwelling arterial and venous catheters. Arterial blood samples were collected immediately before, and at 1, 5, 10, and 30 min after the start of the injection for measurement of the levels of parathyroid hormone (PTH), ionized calcium (Ca²⁺) and compound 17 in plasma. PTH was measured using a specific rat PTH (1-84) ELISA (Immutopics, San Clemente, Calif.) and compound 17 was quantified by an LC-MS/MS method. Positive ions were generated by turbo ionspray and were subsequently fragmented by collision-induced dissociation, so that compound 17 could be detected by selected-reaction monitoring. Plasma levels of compound 17 were maximal at 1 min after the injection and declined rapidly during the next 10-30 min as shown in FIG. 1.

Injection of compound 17 induced a rapid, but transient dose-related increase in plasma PTH levels that were maximal at 1 min after the injection. Plasma PTH levels had returned to pre-dose levels by 10 min after the injection as shown in FIG. 2. There were no consistent changes in plasma Ca²⁺ levels during this experiment (not shown).

Structure I substituted 3H-quinazolin-4-one derivatives have the following chemical formula: 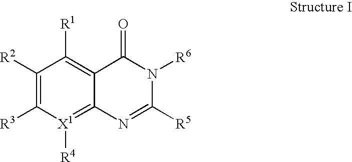
wherein:
R¹, R², and R³ is independently selected from one of: H, halogen, CN, CF₃, OCF₃, lower alkyl, lower alkoxy, NH-acetyl, NH-lower alkyl, NH-alkylaryl, N(lower alkyl)₂, C(O)OH, C(O)O-lower alkyl, C(O)NH-lower alkyl, C(O)N(lower alkyl)₂, OH, OC(O)-lower alkyl, OC(O)-lower alkylamino, OC(O)-lower alkyl-N(lower alk)₂, OP(O)(OH)₂. Preferably, R¹, R², and R³ is each independently selected from one of: H, halogen, lower alkyl, OH, CN, CF₃, OP(O)(OH)₂, lower alkoxy or NH-lower alkyl. More preferably, R¹, R², and R³ is each independently from one of: H, halogen, lower alkyl, OH, CN, CF₃, OP(O)(OH)₂ or NH-lower alkyl;
R⁴ is optional and is selected from one of: H, halogen, CN, CF₃, OCF₃, lower alkyl, lower alkoxy, NH-acetyl, NH-lower alkyl, NH-alkylaryl, N(lower alkyl)₂, C(O)OH, C(O)O-lower alkyl, C(O)NH-lower alkyl, C(O)N(lower alkyl)₂, OH, OC(O)-lower alkyl, OC(O)-lower alkylamino, OC(O)-lower alkyl-N(lower alk)₂, OP(O)(OH)₂. Preferably, R⁴ is selected from one of: H, halogen lower alkyl, OH, CN, CF₃, OP(O)(04₂), lower allcoxy or NH lower alkyl. More preferably, R⁴ is selected from one of: H, halogen, lower alkyl, OH, CN, CF₃, OP(O)(DH)₂ or NH-lower alkyl;
X¹ is selected from one of C and N. Preferably, X¹ is C;
R⁵ is selected from one of: H, lower alkyl, aryl and alkaryl group, which may have 1 to 3 substituents in the aryl ring each selected from one of: H, halogen, CN, CF₃, OCF₃, lower alkyl, NH-alkylaryl, N(lower alkyl)₂, lower alkoxy, OH, OC(O)-lower alkyl, OC(O)-lower alkylamino, OC(O)-lower alkyl-N(lower alkyl)₂, OP(O)(OH)₂. Preferably, R⁵ is furyl or thienyl group, an ethenylphenyl group which may have a substituent in the phenyl ring which is halogen, lower alk, OH, CF₃ or NH-alkylaryl, a pyridyl group which may have 1 to 3 substituents on the pyridyl ring each selected from one of: H, halogen, CN, lower alkyl, lower alkoxy, OH, OC(O)-lower alkylamino, OP(O)(OH)₂, or a phenyl group which may have 1 to 3 substituents on the phenyl ring each selected from one of: H, halogen, CN, CF₃, OCF₃, lower alkyl, NH-lower alkyl, N(lower alkyl)₂, lower alkoxy, OH, OC(O)-lower alkylamino, OP(O)(OH)₂. More preferably, R⁵ is a furyl group, a pyridyl group which may have 1 to 3 substituents on the pyridyl ring which are each independently either H, OH, lower alkyl, halogen, OC(O)-lower alkyl, OC(O)-lower alkyl-N(lower alkyl)₂ or OP(O)(OH)₂, or a phenyl group which may have 1 to 3 substituents on the phenyl ring which are each independently either H, OH, lower alkyl, halogen, OC(O)-lower alkyl, OC(O)-lower alkyl-N(lower alkyl)₂ or OP(O)(OH)₂;
R⁶ is selected from one of: H, lower alkyl and a group of the formula —(CH₂)ₙ—X²—R⁷ wherein n is 0, 1, or 2, X² is lower alk, O, C(O), CH(OH) or a single bond, R⁷ is an aryl group which may have 1 to 3 substituents on the aryl ring each selected from one of: H, halogen, CN, CF₃, OCF₃, lower alkyl, lower alkoxy, NH-lower alkyl, NH-alkylaryl, N(lower alkyl)₂, OH, OC(O)-lower alk, OC(O)-lower alkylamino, OC(O)-lower alkyl-N(lower alk)₂, OP(O)(OH)₂. Preferably, R⁶ is a group of the formula —(CH₂)ₙ—X²—R⁷ wherein n is 0, 1, or 2, X² is O, C(O), CH(OH) or a single bond, R⁷ is a pyridyl group which may have 1 to 3 substituents on the pyridyl ring each selected from one of: H, halogen, CN, CF₃, OCF₃, lower alkyl, lower alkoxy, NH-lower alkyl, NH-alkylaryl, N(lower alkyl)₂, OH, OC(O)-lower alkyl, OC(O)-lower alkylamino, OC(O)-lower alkyl-N(lower alkyl)₂, OP(O)(OH)₂, or a phenyl group which may have 1 to 3 substituents on the phenyl ring each selected from one of: H, halogen, CN, CF₃, OCF₃, lower alkyl, lower alkoxy, NH-lower alkyl, NH-aralkyl, N(lower alkyl)₂, OH, OC(O)-lower alkyl, OC(O)-lower alkylamino, OC(O)-lower alkyl-N(lower alkyl)₂, OP(O)(OH)₂. More preferably, R⁶ is a group of formula —(CH₂)—X²—R⁷ wherein n is 1 or 2, X² is O, C(O), CH(OH) or a single bond, R⁷ is a phenyl group which may have 1 to 3 substituents on the phenyl ring each selected from one of: H, halogen, CN, CF₃, OCF₃, lower alkyl or lower alkoxy;
and pharmaceutically acceptable salts and complexes thereof.

The calcilytic compounds of Structure I described by the present invention can be prepared according to Scheme I using standard techniques. Alternatively, the calcilytic compounds of Structure I may be prepared by N-acylation of 2,4,5,6,7,8-substituted benzo[d][1,3]oxazin-4-ones with primary amines under microwave irradiation conditions. The chemical synthesis for compounds of Structure I by microwave-assisted N-acylation is a novel approach to 2,3,5,6,7,8-substituted 3H-quinazolin-4-ones. A choice of the experimental method was based on availability of the starting materials, and/or on a stability of the substituents in the starting, intermediate, or final compounds under the reaction conditions.

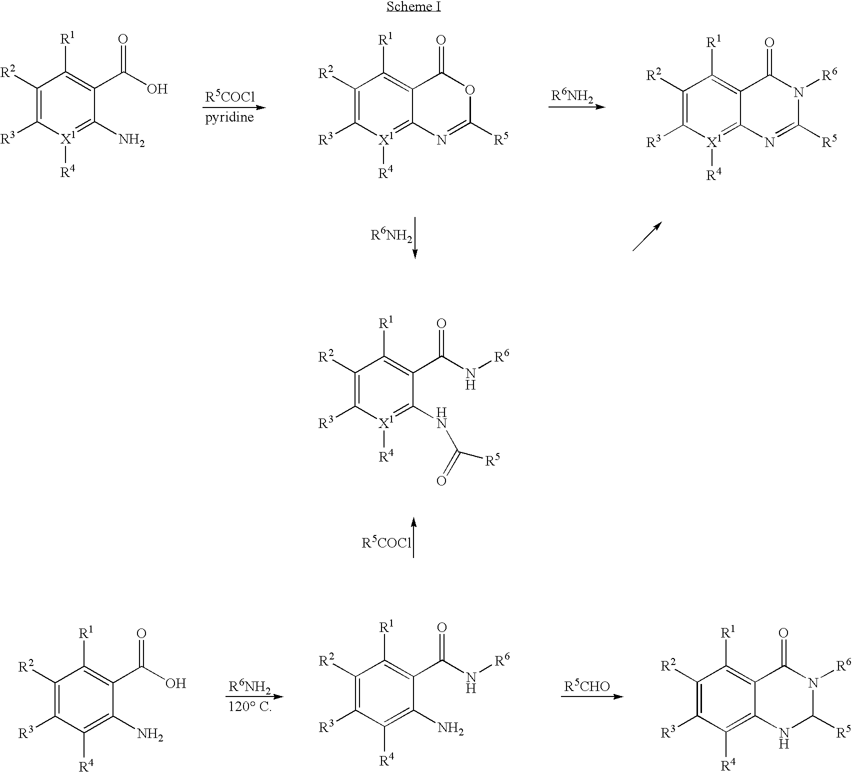

The following specific examples are included for illustrative purposes only and are not to be considered as limiting to this disclosure. The reagents and intermediates used in the following examples are either commercially available or can be prepared according to standard literature procedures by those skilled in the art of organic synthesis.

Microwave reactions were performed on EmryS™ Optimizer (Personal Chemistry, Inc,. Uppsala, Sweden) on continuous irradiation at 2450 MHz. All microwave reactions were carried out in heavy-walled Pyrex tubes, inner diameter 9 mm and height 147 mm, sealed with screw cap fitted Teflon Septa.

GC/EI-MS (Gas Chromatographic/Electron-Impact Mass Spectrometric) analyses were performed on HP-5890 Series gas chromatographs equipped with HP-Ultra-2 columns (30 mm×0.25 mm ID), and HP5971 or HP-5972 Mass Selective Spectrometric Detectors (MSD's) were used.

NMR (Nuclear Magnetic Resonance) spectroscopy was performed on a Varian Gemini 300 spectrometer. Proton and carbon spectra were recorded at 300 MHz and 75 MHz, respectively in deuterochloroform (CDCl₃) or dimethylsulfoxide-d₆ (DMSO-d₆) solutions. NMR resonances are reported in δ (ppm) relative to tetramethylsilane (TMS) as internal standard with the following descriptors for the observed multiplicities: s (singlet), d (doublet), t (triplet), q (quartet), dd (doublet of doublets), and m (multiplet). JAB coupling constants are reported in Hz.

### EXAMPLE 1

### Preparation of 3-Phenethyl-2-phenyl-3H-quinazolin-4-one (2)

### 2-Phenyl-benzo[d][1,3]oxazin-4-one (1)

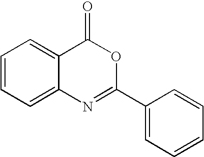

Method A. To a solution of anthranilic acid (1.37 g, 0.01 mol) in pyridine (30 mL) was added benzoyl chloride (2.8 g, 0.02 mol). The mixture was stirred at room temperature for 2 h, and then was poured into ice-cold water (200 mL) under vigorous stirring. After 2 h, the precipitate was filtered off, washed free of pyridine with cold water (3×60 mL) and dried at room temperature. The final product was recrystallized from ethanol to give 1.78 g (80%) of (1) as pale yellow crystals. GC/EI-MS, m/z (rel. int.) 223 (M⁺, 100), 195 (4), 179 (84), 146 (30), 105 (68), 90 (26), 77 (63), 63 (10), 51 (25); RT=7.368 min.

Method B. 2-Benzoylamino-N-(2-Phenylethyl)benzamide (0.34 g, 0.001 mol) was added to polyphosphoric acid (4 g) and heated at 160° C. under stirring for 4 h. The reaction mixture was poured onto ice (10 g) and aqueous ammonia was added to pH 6. The residue was filtered off, washed with water (2×10 ml), dried and recrystallized from ethanol to give 0.11 g (50%) of pale yellow crystals. GC/EI-MS of the compound was identical to that of the product prepared by Method A.

### 3-Phenethyl-2-phenyl-3H-quinazolin-4-one (2)

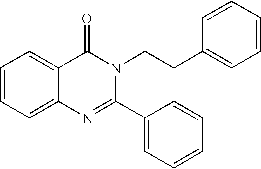

A mixture of 2-phenyl-benzo[d][1,3]oxazin-4-one (1) (0.22 g, 0.001 mol) and phenethylamine (1 mL) was heated at 200° C. under stirring for 2 h. The mixture was poured into a mixture of ice (10 g) and 10% aqueous HCl (10 mL) and vigorously stirred. Diethyl ether was added (5 mL), and the mixture was vigorously stirred again. The upper ethereal layer was allowed to evaporate at room temperature, and the product was filtered off, washed with water, and recrystallized from ethanol to give 0.28 g (88%) colorless crystals of (2). GC/EI-MS, m/z (rel. int.) 326 (M⁺, 2), 235 (16), 222 (100), 295 (7), 178 (6), 152 (4), 119 (41), 104 (12), 77 (20), 51 (6); RT=10.966 min.

In a similar manner, the following quinazolin-4-[3H]-ones were prepared:

### 3-Phenethyl-2-thiophen-2-yl-3H-quinazolin-4-one (3)

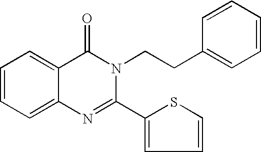

Prepared from 2-thiophen-2-yl-benzo[d][1,3]oxazin-4-one (0.23 g, 0.001 mol) and phenethylamine (1 mL) yielding 0.20 g (60%) of (3) as pale yellow crystals. GC/EI-MS, m/z (rel. int.) 332 (M⁺, 3), 241 (8), 228 (100), 211 (5), 185 (3), 119 (7), 97 (9), 91 (8), 77 (13), 51 (4); RT=10.686 min.

### 2-(3-Fluoro-phenyl)-3-phenethyl-3H-quinazolin-4-one (4)

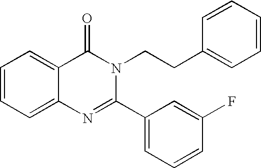

Prepared from 2-(3-fluoro-phenyl)-benzo[d][1,3]oxazin-4-one (0.24 g, 0.001 mol) and phenethylamine (1 mL) yielding 0.25 g (74%) of (4) as colorless crystals. GC/EI-MS, m/z (rel. int.) 344 (M⁺, 21), 253 (91), 241 (100), 223 (40), 196 (29), 170 (14), 119 (100), 104 (74), 77 (67), 65 (18), 50 (23); RY=10.432 min.

### 3-Phenethyl-2-m-tolyl-3H-quinazolin-4-one (5)

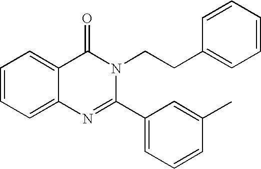

Prepared from 2-(3-methyl-phenyl)-benzo[d][1,3]oxazin-4-one (0.24 g, 0.001 mol) and phenethylamine (1 mL) yielding 0.18 g (53%) of (5) as colorless crystals. GC/EI-MS, m/z (rel. int.) 340 (M⁺, 5), 249 (25), 236 (100), 219 (7), 165 (6), 119 (71), 91 (17), 77 (17), 51 (5); RT=10.956 min.

### 2-(4-Chloro-phenyl)-3-phenethyl-3H-quinazolin-4-one (6)

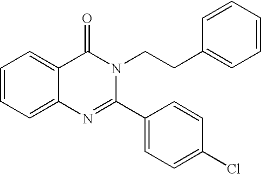

Prepared from 2-(4-chloro-phenyl)-benzo[d][1,3]oxazin-4-one (0.26 g, 0.001 mol) and phenethylamine (1 mL) yielding 0.18 g (50%) of (6) as pale yellow crystals. GC/EI-MS, m/z (rel. int.) 360 (M⁺, 3), 269 (4), 259 (9), 256 (100), 234 (39), 206 (7), 178 (7), 119 (53), 77 (22), 51 (6); RT=11.287 min.

### 2-(3-Chloro-phenyl)-3-phenethyl-3H-quinazolin-4-one (7)

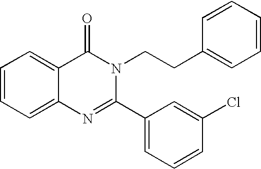

Prepared from 2-(3-chloro-phenyl)-benzo[d][1,3]oxazin-4-one (0.26 g, 0.001 mol) and phenethylamine (1 mL) yielding 0.28 g (80%) of (7) as yellow crystals. GC/EI-MS, m/z (rel. int.) 360 (M⁺, 4), 269 (3), 256 (100), 233(10), 205 (5), 178 (5), 151 (3), 119 (30), 91 (8), 77(15), 51 (4); RT=11.166 min.

### 2-Furan-2-yl-3-[2-(4-methoxy-phenyl)-ethyl]-3H-quinazolin-4-one (8)

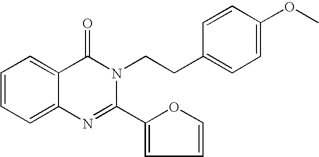

Prepared from 2-furan-2-yl-benzo[d][1,3]oxazin-4-one (0.21 g, 0.001 mol) and 2-(4-methoxy-phenyl)-ethylamine (1 mL) yielding 0.1 g (30%) of (8) as colorless crystals. GC/EI-MS, m/z (rel. int.) 346 (M⁺, 8), 121 (38), 197 (6), 169 (3), 134 (100), 121 (17), 91 (9), 77 (12), 51 (3); RT=11.028 min.

### 3-Phenethyl-2-p-tolyl-3H-quinazolin-4-one (9)

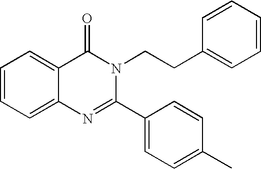

Prepared from 2-(p-tolyl)-benzo[d][1,3]oxazin-4-one (0.23 g, 0.001 mol) and phenethylamine (1 mL) yielding 0.2 g (60%) of (9) as colorless crystals. GC/EI-MS, m/z (rel. int.) 340 (M⁺, 3), 249 (23), 236 (100), 220 (2), 192 (6), 165 (5), 119 (47), 104 (7), 77 (13), 65 (5), 51 (4); RT=11.014 min.

### 2-(4-Methoxy-phenyl)-3-phenethyl-3H-quinazolin-4-one (10)

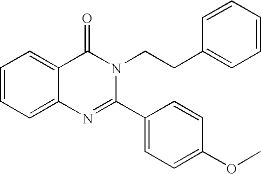

Prepared from 2-(4-methoxy-phenyl)-4H-3,1-benzoxazin-4-one (0.25 g, 0.001 mol) and phenethylamine (1 mL) yielding 0.17 g (50%) of (10) as colorless crystals. ¹H NMR (CDCl₃): δ 8.36 (1H, d, J=8.0), 7.77-7.71 (2H, m), 7.54-7.51 (1H, m), 7.49-7.31 (2H, m), 7.26-7.18 (3H, m), 7.02-6.98 (2H, m), 6.94-6.91 (2H, m), 4.24 (2H, t, J=7.7), 2.92 (2H, t, J=7.6). GC/EI-MS, m/z (rel. int.) 356 (M⁺, 4), 265 (9), 252 (100), 221 (9), 166 (4), 139 (3), 119 (28), 91 (6), 77 (10), 51 (2); RT=11.617 min.

### 2-(3-Methoxy-phenyl)-3-phenethyl-3H-quinazolin-4-one (11)

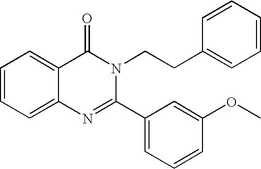

Prepared from 2-(3-methoxy-phenyl)-4H-3,1-benzoxazin-4-one (0.25 g, 0.001 mol) and phenethylamine (1 mL) yielding 0.28 g (80%) of (11) as colorless crystals. ¹H NMR (CDCl₃): δ 8.37 (1H, d, J=8.8), 7.78-7.73 (2H, m), 7.56-7.50 (1H, m), 7.41 (1H, t, J=8.0), 7.26-7.17 (3H, m), 7.06 (1H, dd, J=8.2, 2.5), 6.97 (1H, d, J=8.5), 6.94-6.91 (3H, m), 4.22-4.17 (2H, m), 3.84 (3H, s), 2.97-2.92 (2H, m). GC/EI-MS, m/z (rel. int.) 356 (M⁺, 4) 265 (12), 252 (100), 193 (4), 166 (3), 139 (5), 119 (10), 104 (10), 91 (10), 77 (15), 51 (4); RT=11.452 min.

### 2-(2-Chloro-phenyl)-3-phenethyl-3H-quinazolin-4-one (12)

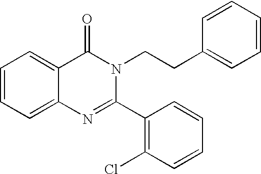

Prepared from 2-(2-chloro-phenyl)-4H-3,1-benzoxazin-4-one (0.26 g, 0.001 mol) and phenethylamine (1 mL) yielding crude (11) which was purified by column chromatography on silica gel (eluent CHCl₃-EtOAc, 10:1, R_{f} 0.7) to give 0.15 g (42%) of (12) as pale yellow crystals. GC/EI-MS, m/z (rel. int.) 360 (M⁺, 6), 325 (99), 269 (5), 256 (100), 234 (61), 221 (9), 206 (10), 178 (11), 151 (6), 119 (78), 104 (24), 7 (22), 51 (6); RT=11.120 min.

### 3-Phenethyl-2-o-tolyl-3H-quinazolin-4-one (13)

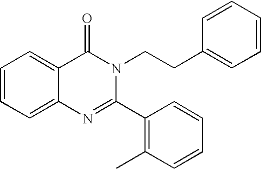

Prepared from 2-(o-tolyl)-benzo[d][1,3]oxazin-4-one (0.24 g, 0.001 mol) and phenethylamine (1 mL) yielding a crude oil of (12) which was purified by column chromatography on silica gel (eluent CHCl₃—EtOAc, 15:1, R_{f} 0.75) to give 0.2 g (59%) of pure (13) as colorless crystals. ¹H NMR (CDCl₃): δ 8.41-8.37 (2H, m), 7.82-7.74 (2H, m), 7.57-7.52 (1H, m), 7.47-7.41 (1H, m), 7.36-7.31 (2H, m), 7.26-7.16 (2H, m), 6.88-6.84 (2H, m), 4.45-4.36 (2H, m), 3.69-3.59 (2H, m), 3.00-2.91 (2H, m), 2.85-2.75 (2H, m), 2.21 (3H, s). ¹³C NMR (CDCl₃): δ 161.96, 155.69, 147.27, 137.69, 135.32, 134.71, 134.36, 130.56, 129.86, 128.72, 128.54, 127.75, 127.52, 127.06, 126.67, 126.59, 126.19, 120.98, 41.14, 34.50, 19.18. GC/EI-MS, m/z (rel. int.) 340 (M⁺, 17), 325 (24), 249 (27), 236 (100), 235 (100), 219 (8), 207 (10), 165 (7), 119 (15), 104 (10), 91 (10), 77 (12), 51 (3); RT=10.733 min.

### 2-Furan-2-yl-3-[2-(2-methoxy-phenyl)-ethyl]-3H-quinazolin-4-one (14)

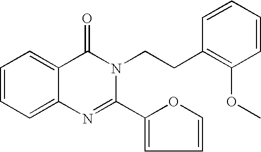

Prepared from 2-furan-2-yl-benzo[d][1,3]oxazin-4-one (0.21 g, 0.001 mol) and 2-(2-methoxy-phenyl)-ethylamine (1 mL) yielding 0.18 g (55%) of (14) as colorless crystals. GC/EI-MS, m/z (rel. int.) 346 (M⁺, 24), 329 (8), 212 (100), 197 (13), 134 (35), 119 (27), 91 (27), 77 (13), 65 (6), 51 (4); RT=10.763 min.

### 3-Phenethyl-2-pyridin-4-yl-3H-quinazolin-4-one (15)

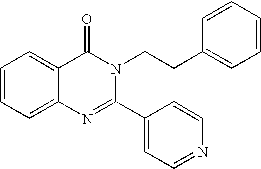

Prepared from 2-pyridin-4-yl-benzo[d][1,3]oxazin-4-one (0.22 g, 0.001 mol) and phenethylamine (1 mL) yielding 0.16 g (50%) of (15) as pale crystals. GC/EI-MS, m/z (rel. int.) 327 (M⁺, 73), 236 (36), 223 (90), 207 (12), 179 (9), 119 (43), 104 (100), 77 (27), 65 (5), 51 (6); RT=10.818 min.

### 2-Methyl-3-phenethyl-3H-quinazolin-4-one (16)

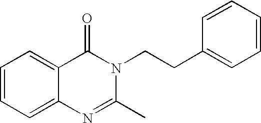

Prepared from 2-methyl-benzo[d][1,3]oxazin-4-one (6.44 g, 0.04 mol) and phenethylamine (6 mL) yielding 5.2 g (50%) of (16) as pale needles. GC/EI-MS, m/z (rel. int.) 264 (M⁺, 13), 173 (8), 160 (100), 144 (48), 116 (25), 104 (86), 91 (23), 77 (49), 65 (15), 51 (14); RT=9.048 min.

### EXAMPLE 2

### Preparation of 2-(2-Hydroxy-phenyl)-3-phenethyl-3H-quinazolin-4-one (17)

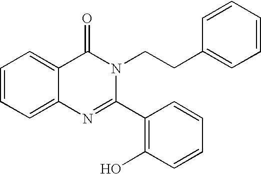

Method A. A mixture of acetic acid 2-(4-oxo-4H-benzo[d][1,3]oxazin-2-yl)-phenyl ester (0.28 g, 0.001 mol) and phenethylamine (1 mL) was heated at 200° C. under stirring until GC/MS control showed no starting materials (about 2 h). The mixture was poured into a mixture of ice (10 g) and 10% aqueous HCl (10 mL) and vigorously stirred. Diethyl ether was added (5 mL), and the mixture was vigorously stirred again. The upper ethereal layer was allowed to evaporate at room temperature, and the oily product was separated, washed with water (5 mL) and dissolved in chloroform (10 mL). The organic solution was dried over anhydrous Na₂SO₄, and the solvent was removed in vacuo. The residue was purified by column chromatography on silica gel (eluent CHCl₃—EtOAc, 7:3, R_{f} 0.5) to afford 0.28 g (80%) of (17) as colorless crystals. GC/EI-MS, m/z (rel. int.) 342 (M⁺, 3), 341 (5), 250 (6), 238 (100), 223 (4), 210 (3), 166 (5), 140 (3), 119 (5), 91 (9), 77 (13), 51 (4). ¹H NMR (DMSO-d₆): δ 9.60 (1H, OH, broad s), 8.04 (1H, dd, J=7.9, 1.4), 7.66 (1H, t, J=8.0), 7.53 (1H, d, J=8.0), 7.38 (1H, t, J=8.1), 7.22-7.09 (5H, m), 6.89 (1H, t, J=8.0), 6.82-6.79 (2H, m), 6.75 (1H, d, J=8.2), 4.22 (2H, t, J=7.8), 2.82 (2H, t, J=7.8). ¹³C NMR (DMSO-d₆): δ 161.5, 155.9, 154.2, 145.3, 137.5, 134.7, 132.1, 129.2, 128.7, 128.5, 127.2, 127.0, 126.6, 125.4, 121.3, 120.2, 118.5, 47.5, 34.5.

Method B. A mixture of 2-(2-methoxy-phenyl)-benzo[d][1,3]oxazin-4-one (0.28 g, 0.001 mol) and phenethylamine (1 mL) was heated at 200° C. under stirring for 4 h. The mixture was worked up as described in Method A, and 0.25 g (70%) of colorless crystals were isolated and ¹H and ¹³C NMR spectral characteristics of the compound were identical to those of the product obtained by Method A.

Method C. A dried heavy-walled Pyrex tube was charged with acetic acid 2-(4-oxo-4H-benzo[d][1,3]oxazin-2-yl)-phenyl ester (0.28 g, 0.001 mol) and phenethylamine (0.242 g, 0.002 mol) in DMF (1 mL). The screw cap was tightened thoroughly. The reaction mixture was exposed to microwave irradiation at 240° C. for 10 min. The reaction tube was allowed to reach room temperature, and the reaction mixture was worked up as described in Method A to give 0.31 g (99%) of colorless crystals. GC/EI-MS of the compound was identical to that of the product prepared by Method A.

In a similar manner, the quinazolin-4-[3H]-one compounds 18-71 were prepared by Method A, compound 43 by Method A and C, and compound 109 by Method C.

### 2-(2,5-Dihydroxy-phenyl)-3-phenethyl-3H-quinazolin-4-one (18)

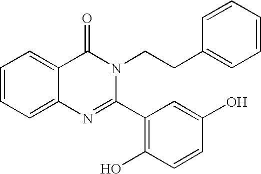

Prepared from acetic acid 4-acetoxy-2-(4-oxo-4H-benzo[d][1,3]oxazin-2-yl)-phenyl ester (0.34 g, 0.001 mol) and phenethylamine (1 mL) yielding 0.28 g (80%) of (18) as colorless crystals after recrystallization from acetone. ¹H NMR (DMSO-d₆): δ 9.41 (1H, OH, broad s)m 9.10 (1H, OH, broad s), 8.21 (1H, d, J=8.0), 7.86 (1H, t, J=8.2), 7.68-7.55 (2H, m), 7.22-7.17 (3H, m), 6.85-6.70 (5H, m), 4.11-4.07 (2H, m), 2.84-2.82 (2H, m). ¹³C NMR (DMSO-d₆): δ 160.99, 154.28, 149.82, 147.22, 146.39, 138.03, 134.37, 128.49, 128.29, 127.15, 126.88, 126.47, 126.12, 122.94, 120.46, 117.85, 116.61, 115.82, 46.32, 33.98. GC/EI-MS, m/z (rel. int.) 358 (M⁺, 6), 267 (2), 254 (100), 197 (5), 136 (6), 119 (13), 105 (8), 77 (12), 51 (3); RT=12.200 min.

### 2-(3-Hydroxy-phenyl)-3-phenethyl-3H-quinazolin-4-one (19)

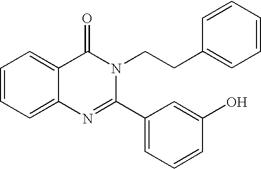

Prepared from acetic acid 3-(4-oxo-4H-benzo[d][1,3]oxazin-2-yl)-phenyl ester (0.56 g, 0.002 mol) and phenethylamine (2 mL) yielding 0.42 g (62%) of (19) as colorless crystals after recrystallization from acetone. ¹H NMR (CDCl₃): δ 8.35 (1H, dd, J=8.0, 1.4), 7.85 (1H, d, J=8.0), 7.84-7.74 (1H, m), 7.57-7.52 (1H, m), 7.27-7.14 (4H, m), 6.92-6.78 (5H, m), 4.18-4.13 (2H, m), 2.93-2.88 (2H, m). ¹³C NMR (CDCl₃): δ 161.43, 157.29, 156.98, 145.42, 137.40, 134.98, 130.01, 128.77, 128.62, 127.68, 126.99, 126.73, 125.92, 120.59, 118.84, 118.37, 115.44, 106.90, 47.88, 34.67. GC/EI-MS, m/z (rel. int.) 342 (M⁺, 3), 250 (5), 238 (100), 119 (38), 91 (9), 77 (14), 51 (4); RT 11.708 min.

### 2-(4-Hydroxy-phenyl)-3-phenethyl-3H-quinazolin-4-one (20)

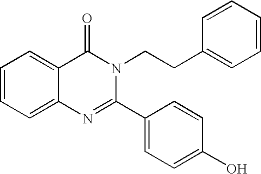

Prepared from acetic acid 4-(4-oxo-4H-benzo[d][1,3]oxazin-2-yl)-phenyl ester (0.56 g, 0.002 mol) and phenethylamine (2 mL) yielding 0.56 g (80%) of (20) as colorless crystals after recrystallization from acetone. ¹H NMR (DMSO-d₆): δ 11.60 (1H, OH, broad s), 8.29 (1H, d, J=8.0), 8.04-7.94 (2H, m), 7.75-7.71 (1H, m), 7.55-7.52 (2H, m), 7.24-7.20 (3H, m), 7.09-7.06 (2H, m), 6.96-6.93 (2H, m), 4.17-4.12 (2H, m), 2.93-2.88 (2H, m). ¹³C NMR (DMSO-d₆): δ 160.45, 160.05, 158.75, 140.99, 137.50, 135.72, 130.26, 128.61, 128.40, 128.31, 126.82, 126.67, 122.54, 120.47, 119.37, 115.47, 47.68, 33.45. GC/EI-MS, m/z (rel. int.) 342 (M⁺, 2), 251 (15), 238 (100), 166 (5) 119 (38), 77 (15), 65 (7), 51 (4); RT=11.883 min.

### 2-(2-Hydroxy-phenyl)-3-[2-(4-hydroxy-phenyl)-ethyl]-3H-quinazolin-4-one (21)

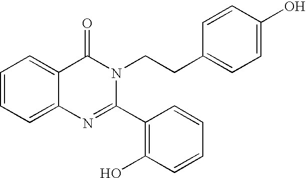

Prepared from acetic acid 2-(4-oxo-4H-benzo[d][1,3]oxazin-2-yl)-phenyl ester (0.56 g, 0.002 mol) and tyramine (0.69 g, 0.005 mol) yielding 0.62 g (87%) of (21) as colorless crystals after recrystallization from acetone. ¹H NMR (DMSO-d₆): δ 11.10 (2H, OH, broad s), 8.30 (1H, d, J=8.0), 8.04-7.98 (1H, m), 7.89 (1H, d, J=8.2), 7.74 (1H, t, J=7.5), 7.59-7.47 (1H, m), 7.26 (1H, d, J=8.2), 7.08 (1H, t, J=7.5), 6.34 (4H, s), 4.06-4.01 (2H, m), 2.77-2.71 (2H, m). ¹³C NMR (DMSO-d₆): δ 159.76, 156.79, 156.22, 154.59, 141.61, 135.78, 132.99, 129.76, 129.28, 128.56, 127.42, 126.93, 123.05, 119.63, 119.28, 118.17, 116.39, 115.47, 47.57, 32.85. GC/EI-MS, m/z (rel. int.) 358 (M⁺, 1), 357 (2)251 (6), 239 (100), 223 (4), 166 (6), 119 (26), 91 (11), 77 (19), 51 (3); RT=12.213 min.

### 2-(2-Hydroxy-phenyl)-3-(2-phenoxy-ethyl)-3H-quinazolin-4-one (22)

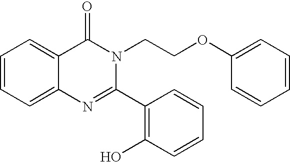

Prepared from acetic acid 2-(4-oxo-4H-benzo[d][1,3]oxazin-2-yl)-phenyl ester (0.56 g, 0.002 mol) and 2-phenoxy-ethylamine (1.5 mL) yielding 0.42 g (60%) of (22) as colorless crystals after recrystallization from ethanol. ¹H NMR (DMSO-d₆): δ 8.70 (1H, OH, broad s), 8.28 (1H, dd, J=8.2, 1.1), 8.00-7.95 (1H, m), 7.85 (1H, d, J=8.2), 7.22-7.67 (1H, m), 7.61 (1H, dd, J=7.7, 1.6), 7.53-7.47 (1H, m), 7.23-7.18 (3H, m), 7.07 (1H, t, J=7.4), 6.88 (1H, t, J=7.2), 6.76-6.73 (2H, m), 4.35 (2H, broad t, J=5.2), 4.15 (2H, t, J=5.7). GC/EI-MS, m/z (rel. int.) 358 (M⁺, 3), 265 (45), 238 (100), 221 (5), 166 (5), 146 (4), 119 (15), 91 (6), 77 (11) 65 (8), 51 (4); RT=11.442 min.

### 3-[2-(4-Fluoro-phenyl)-ethyl]-2-(2-hydroxy-phenyl)-3H-quinazolin-4-one (23)

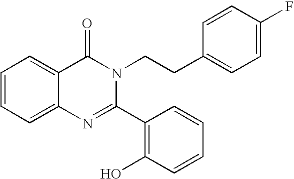

Prepared from acetic acid 2-(4-oxo-4H-benzo[d][1,3]oxazin-2-yl)-phenyl ester (0.28 g, 0.001 mol) and 2-(4-fluoro-phenyl)-ethylamine (1 mL) yielding 0.29 g (80%) of (23) as pale crystals after recrystallization from ethanol. GC/EI-MS, m/z (rel. int.) 360 (M⁺, 2), 359 (4), 250 (5), 238 (100), 223 (4), 166 (4), 7 (7), 51 (2); RT=10.864 min.

### 3-[2-(3-Fluoro-phenyl)-ethyl]-2-(2-hydroxy-phenyl)-3H-quinazolin-4-one (24)

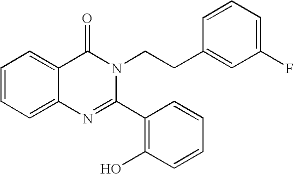

Prepared from acetic acid 2-(4-oxo-4H-benzo[d][1,3]oxazin-2-yl)-phenyl ester (0.28 g, 0.001 mol) and 2-(3-fluoro-phenyl)-ethylamine (1 mL) yielding 0.29 g (80%) of (24) as pale crystals after recrystallization from ethanol. GC/EI-MS, m/z (rel. int.) 360 (M⁺, 6), 359 (8), 250 (9), 238 (100), 223 (5), 166 (5), 77 (8), 51 (2); RT=10.877 min.

### 3-[2-(2-Fluoro-phenyl)-ethyl]-2-(2-hydroxy-phenyl)-3H-quinazolin-4-one (25)

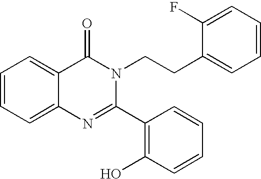

Prepared from acetic acid 2-(4-oxo-4H-benzo[d][1,3]oxazin-2-yl)-phenyl ester (0.28 g, 0.001 mol) and 2-(2-fluoro-phenyl)-ethylamine (1 mL) yielding 0.27 g (75%) of (25) as pale crystals after recrystallization from ethanol. GC/EI-MS, m/z (rel. int.) 360 (M⁺, 8), 359 (10), 251 (6), 238 (100), 210 (4), 166 (5), 119 (20), 92 (6), 77 (8), 51 (2); RT=10.867 min.

### 3-[2-(4-Chloro-phenyl)-ethyl]-2-(2-hydroxy-phenyl)-3H-quinazolin-4-one (26)

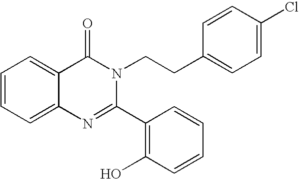

Prepared from acetic acid 2-(4-oxo-4H-benzo[d][1,3]oxazin-2-yl)-phenyl ester (0.28 g, 0.001 mol) and 2-(4-chloro-phenyl)-ethylamine (1 mL) yielding 0.23 g (60%) of (26) as pale yellow crystals after recrystallization from ethanol. GC/EI-MS, m/z (rel. int.) 376 (M⁺, 1), 375 (3), 250 (5), 238 (100), 210 (3) 166 (4), 119 (20), 92 (5), 77 (9), 51 (2); RT=11.651 min.

### 3-[2-(3-Chloro-phenyl)-ethyl]-2-(2-hydroxy-phenyl)-3H-quinazolin-4-one (27)

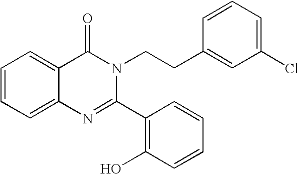

Prepared from acetic acid 2-(4-oxo-4H-benzo[d][1,3]oxazin-2-yl)-phenyl ester (0.28 g, 0.001 mol) and 2-(3-chloro-phenyl)-ethylamine (1 mL) yielding 0.31 g (83%) of (27) as pale yellow crystals after recrystallization from ethanol. GC/EI-MS, m/z (rel. int.) 376 (M⁺, 5), 375 (7), 250 (9), 238 (100), 210 (4), 166 (6), 140 (4), 119 (26), 92 (8), 77 (12), 51 (3); RT=11.611 min.

### 3-[2-(2-Chloro-phenyl)-ethyl]-2-(2-hydroxy-phenyl)-3H-quinazolin-4-one (28)

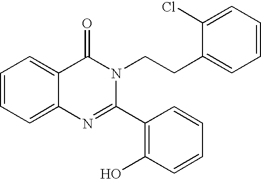

Prepared from acetic acid 2-(4-oxo-4H-benzo[d][1,3]oxazin-2-yl)-phenyl ester (0.28 g, 0.001 mol) and 2-(2-chloro-phenyl)-ethylamine (1 mL) yielding 0.30 g (80%) of (28) as pale yellow crystals after recrystallization from ethanol. GC/EI-MS, m/z (rel. int.) 376 (M⁺, 6), 375 (7), 251 (8), 238 (100), 210 (4), 166 (6), 139 (5), 119 (25), 92 (9), 77 (16), 51 (3); RT=11.554 min.

### 2-(2-Hydroxy-phenyl)-3-[2-(4-methoxy-phenyl)-ethyl]-3H-quinazolin-4-one (29)

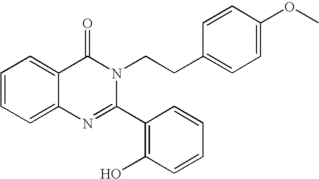

Prepared from acetic acid 2-(4-oxo-4H-benzo[d][1,3]oxazin-2-yl)-phenyl ester (0.28 g, 0.001 mol) and 2-(4-methoxy-phenyl)-ethylamine (1 mL) yielding 0.29 g (78%) of (29) as pale crystals after recrystallization from ethanol. GC/EI-MS, m/z (rel. int.) 372 (M⁺, 0.2), 371 (0.6), 250 (2), 238 (100), 166 (4), 134 (23), 119 (20), 91 (11), 77 (11), 51 (2); RT=11.801 min.

### 2-(2-Hydroxy-phenyl)-3-[2-(3-methoxy-phenyl)-ethyl]-3H-quinazolin-4-one (30)

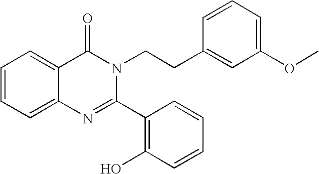

Prepared from acetic acid 2-(4-oxo-4H-benzo[d][1,3]oxazin-2-yl)-phenyl ester (0.28 g, 0.001 mol) and 2-(3-methoxy-phenyl)-ethylamine (1 mL) yielding 0.28 g (75%) of (30) as pale crystals after recrystallization from ethanol. GC/EI-MS, m/z (rel. int.) 372 (M⁺, 2), 371 (3), 250 (5), 238 (100), 210 (3), 166 (4), 134 (10), 119 (21), 91 (11), 77 (10), 51 (2); RT=11.691 min.

### 2-(2-Hydroxy-phenyl)-3-[2-(2-methoxy-phenyl)-ethyl]-3H-quinazolin-4-one (31)

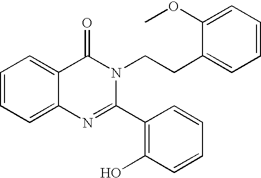

Prepared from acetic acid 2-(4-oxo-4H-benzo[d][1,3]oxazin-2-yl)-phenyl ester (0.28 g, 0.001 mol) and 2-(2-methoxy-phenyl)-ethylamine (1 mL) yielding 0.31 g (82%) of (31) as pale crystals after recrystallization from ethanol. GC/EI-MS, m/z (rel. int.) 372 (M⁺, 1), 371 (2), 251 (3), 238 (100), 210 (2), 166 (5), 119 (24), 91 (22), 77 (11), 51 (3); RT=11.460 min.

### 2-(2-Hydroxy-phenyl)-3-(4-methyl-benzyl)-3H-quinazolin-4-one (32)

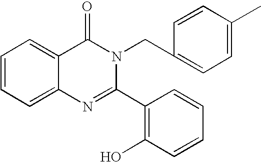

Prepared from acetic acid 2-(4-oxo-4H-benzo[d][1,3]oxazin-2-yl)-phenyl ester (0.28 g, 0.001 mol) and 4-methyl-benzylamine (1 mL) yielding 0.29 g (78%) of (32) as colorless crystals after recrystallization from ethanol. GC/EI-MS, m/z (rel. int.) 342 (M⁺, 36), 341 (69), 325 (66), 238 (28), 119 (18), 105 (100), 91 (16), 77 (30), 51 (6); RT=10.946 min.

### 2-(2-Hydroxy-phenyl)-3-(2-p-tolyl-ethyl)-3H-quinazolin-4-one (33)

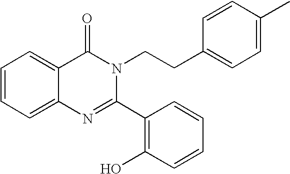

Prepared from acetic acid 2-(4-oxo-4H-benzo[d][1,3]oxazin-2-yl)-phenyl ester (0.28 g, 0.001 mol) and 2-p-tolyl-ethylamine (1 mL) yielding 0.29 g (81%) of (33) as colorless crystals after recrystallization from ethanol. GC/EI-MS, m/z (rel. int.) 356 (M⁺, 1), 355 (2), 238 (100), 223 (3), 166 (5), 119 (20), 91 (9), 77 (11), 51 (2); RT=11.154 min.

### 3-[2-(3,4-Dichloro-phenyl)-ethyl]-2-(2-hydroxy-phenyl)-3H-quinazolin-4-one (34)

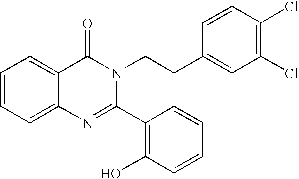

Prepared from acetic acid 2-(4-oxo-4H-benzo[d][1,3]oxazin-2-yl)-phenyl ester (0.28 g, 0.001 mol) and 2-(3,4-dichloro-phenyl)-ethylamine (1 mL) yielding 0.29 g (70%) of (34) as pale yellow crystals after recrystallization from ethanol. GC/EI-MS, m/z (rel. int.) 411 (M⁺, 3), 410 (2), 409 (4), 250 (7), 238 (100), 210 (3), 159 (4), 119 (22), 91 (4), 77 (7), 51 (2); RT=12.242 min.

### 3-[2-(2,4-Dichloro-phenyl)-ethyl]-2-(2-hydroxy-phenyl)-3H-quinazolin-4-one (35)

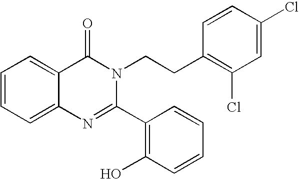

Prepared from acetic acid 2-(4-oxo-4H-benzo[d][1,3]oxazin-2-yl)-phenyl ester (0.28 g, 0.001 mol) and 2-(2,4-dichloro-phenyl)-ethylamine (1 mL) yielding 0.27 g (65%) of (35) as pale yellow crystals after recrystallization from ethanol. GC/EI-MS, m/z (rel. int.) 411 (M⁺, 4), 410 (4), 409 (5), 251 (8), 238 (100), 159 (6), 119 (22), 92 (8), 77 (8), 51 (3); RT=12.534 min.

### 3-[2-(3,4-Dimethoxy-phenyl)-ethyl]-2-(2-hydroxy-phenyl)-3H-quinazolin-4-one (36)

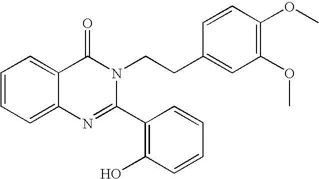

Prepared from acetic acid 2-(4-oxo-4H-benzo[d][1,3]oxazin-2-yl)-phenyl ester (0.28 g, 0.001 mol) and 2-(3,4-dimethoxy-phenyl)-ethylamine (1 mL) yielding 0.20 g (50%) of (36) as colorless crystals after recrystallization from ethanol. GC/EI-MS, m/z (rel. int.) 402 (M⁺, 1), 401 (1), 250 (4), 238 (100), 164 (85), 149 (12), 119 (22), 91 (12) 77 (15), 51 (3); RT=12.478 min.

### 2-(2-Hydroxy-phenyl)-6-methyl-3-phenethyl-3H-quinazolin-4-one (37)

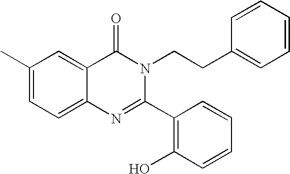

Prepared from acetic acid 2-(6-methyl-4-oxo-4H-benzo[d][1,3]oxazin-2-yl)-phenyl ester (0.29 g, 0.001 mol) and phenethylamine (1 mL) yielding 0.28 g (80%) of (37) as colorless crystals after recrystallization from ethanol. ¹H NMR (DMSO-d₆): δ 10.80 (1H, OH, broad s), 8.08 (1H, s), 7.82-7.73 (2H, m), 7.51 (1H, t, J=7.7), 7.37 (1H, t, J=7.4), 7.21-7.19 (4H, m), 7.02 (1H, t, J=7.4), 6.83-6.81 (2H, m), 4.07 (2H, broad t, J=7.1), 2.83 (2H, t, J=7.7), 2.52 (3H, s). ¹³C NMR (DMSO-d₆): δ 159.92, 155.35, 154.48, 138.17, 137.59, 136.65, 132.46, 129.76, 128.57, 128.34, 126.58, 125.97, 123.84, 119.58, 119.17, 116.15, 46.94, 33.65, 20.91. GC/EI-MS, m/z (rel. int.) 356 (M⁺, 4), 355 (6), 264 (6), 252 (100), 133 (22), 104 (8), 91 (8), 77 (7), 51 (2); RT=11.318 min.

### 6-Fluoro-2-(2-hydroxy-phenyl)-3-phenethyl-3H-quinazolin-4-one (38)

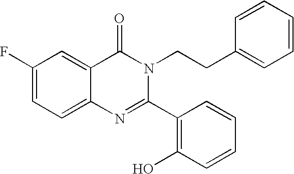

Prepared from acetic acid 2-(6-fluoro-4-oxo-4H-benzo[d][1,3]oxazin-2-yl)-phenyl ester (0.30 g, 0.001 mol) and phenethylamine (1 mL) yielding 0.27 g (74%) of (38) as colorless crystals after recrystallization from ethanol. ¹H NMR (DMSO-d₆): δ 10.24 (1H, OH, broad s), 7.92-7.88 (1H, m), 7.78-7.75 (2H, m), 7.45-7.40 (1H, m), 7.26-7.17 (4H, m), 7.05 (1H, d, J=8.0), 6.96 (1H, t, J=7.4), 6.80-6.77 (2H, m), 4.03 (2H, broad t, J=7.4), 2.80 (2H, t, J=7.7). ¹³C NMR (DMSO-d₆): δ 161.83, 160.37, 160.32, 158.58, 154.12, 153.95, 143.90, 137.85, 131.27, 130.02, 129.90, 129.77, 128.51, 128.31, 126.47, 123.22, 122.89, 122.32, 212.68, 121.56, 119.09, 115.72, 110.89, 110.58, 46.55, 33.79. GC/EI-MS, m/z (rel. int.) 360 (M⁺, 2), 359 (3), 268 (5), 256 (100), 184 (6), 137 (28), 104 (6), 91 (10), 77 (8), 65 (5), 51 (3); RT=10.699 min.

### 6-Chloro-2-(2-hydroxy-phenyl)-3-phenethyl-3H-quinazolin-4-one (39)

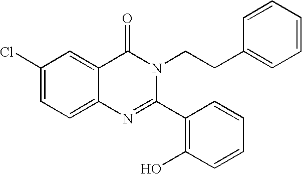

Prepared from acetic acid 2-(6-chloro-4-oxo-4H-benzo[d][1,3]oxazin-2-yl)-phenyl ester (0.32 g, 0.001 mol) and phenethylamine (1 mL) yielding 0.28 g (75%) of (39) as pale yellow crystals after recrystallization from ethanol. GC/EI-MS, m/z (rel. int.) 376 (M⁺, 3), 375 (4), 285 (4), 274 (3), 272 (100), 257 (4), 153 (21), 104 (5), 91 (6), 77 (5), 51 (1); RT=11.704 min.

### 2-(2-Hydroxy-phenyl)-5-methyl-3-phenethyl-3H-quinazolin-4-one (40)

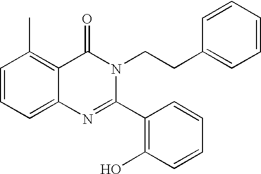

Prepared from acetic acid 2-(5-methyl-4-oxo-4H-benzo[d][1,3]oxazin-2-yl)-phenyl ester (0.29 g, 0.001 mol) and phenethylamine (1 mL) yielding 0.28 g (80%) of (40) as colorless crystals after recrystallization from ethanol. GC/EI-MS, m/z (rel. int.) 356 (M⁺, 3), 355 (5), 264 (6), 252 (100), 237 (4), 207 (2), 133 (2), 104 (8), 91 (12), 77 (10), 51 (3); RT=11.046 min.

### 7-Chloro-2-(2-hydroxy-phenyl)-3-phenethyl-3H-quinazolin-4-one (41)

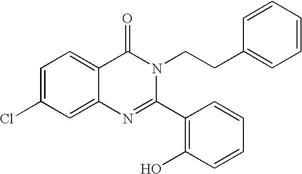

Prepared from acetic acid 2-(7-chloro-4-oxo-4H-benzo[d][1,3]oxazin-2-yl)-phenyl ester (0.32 g, 0.001 mol) and phenethylamine (1 mL) yielding 0.28 g (75%) of (41) as pale yellow crystals after recrystallization from ethanol. GC/EI-MS, m/z (rel. int.) 376 (M⁺, 2), 375 (4), 284 (5), 274 (34), 273 (19), 272 (100), 257 (4), 153 (17), 126 (6), 104 (7), 91 (9), 77 (7), 51 (2); RT=11.539 min.

### 2-(2-Hydroxy-phenyl)-8-methyl-3-phenethyl-3H-quinazolin-4-one (42)

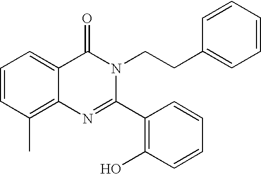

Prepared from acetic acid 2-(8-methyl-4-oxo-4H-benzo[d][1,3]oxazin-2-yl)-phenyl ester (0.29 g, 0.001 mol) and phenethylamine (1 mL) yielding 0.28 g (80%) of (42) as pale crystals after recrystallization from ethanol. GC/EI-MS, m/z (rel. int.) 356 (M⁺, 3), 355 (5), 264 (5), 252 (100), 224 (2), 180 (4), 133 (3), 105 (13), 91 (7), 77 (6), 65 (4), 51 (2); RT=11.102 min.

### 6-Fluoro-3-[2-(3-fluoro-phenyl)-ethyl]-2-(2-hydroxy-phenyl)-3H-quinazolin-4-one (43)

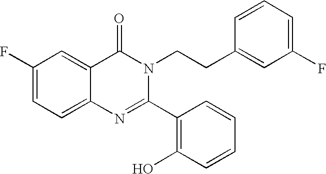

Prepared by Method A from acetic acid 2-(6-fluoro-4-oxo-4H-benzo[d][1,3]oxazin-2-yl)-phenyl ester (0.29 g, 0.001 mol) and 2-(3-fluoro-phenyl)-ethylamine (1 mL) yielding 0.28 g (75%) of (43) as pale crystals after recrystallization from ethanol. Alternatively, prepared by Method C from acetic acid 2-(6-fluoro-4-oxo-4H-benzo[d][1,3]oxazin-2-yl)-phenyl ester (0.29 g, 0.001 mol) and 2-(3-fluoro-phenyl)-ethylamine (0.278 g, 0.002 mol) yielding 0.36 g (99%) of (43) as pale crystals after recrystallization from ethanol. ¹H NMR (DMSO-d₆): δ 10.23 (1H, OH, s), 7.91-7.87 (1H, m), 7.78-7.75 (2H, m), 7.45-7.40 (1H, m), 7.27-7.20 (2H, m), 7.05-6.93 (3H, m), 6.3 (1H, d, J=7.7), 6.59-6.55 (1H, m), 4.06 (2H, t, J=7.5), 2.84 (2H, t, J=7.7). ¹³C NMR (DMSO-d₆): δ 163.79, 161.84, 160.56, 160.40, 158.58, 154.08, 153.81, 144.13, 140.83, 140.73, 131.26, 130.46, 130.35, 130.22, 130.11, 129.80, 124.47, 123.21, 122.89, 122.48, 121.68, 121.57, 119.14, 115.72, 115.24, 114.96, 113.49, 113.20, 110.87, 110.56, 46.22, 33.44. GC/EI-MS, m/z (rel. int.) 378 (M⁺, 6), 377 (6), 268 (8), 256 (100) 241 (5), 184 (5), 137 (25), 109 (7); RT=10.715 min.

### 6-Fluoro-3-[2-(2-fluoro-phenyl)-ethyl]-2-(2-hydroxy-phenyl)-3H-quinazolin-4-one (44)

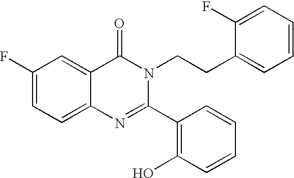

Prepared from acetic acid 2-(6-fluoro-4-oxo-4H-benzo[d][1,3]oxazin-2-yl)-phenyl ester (0.29 g, 0.001 mol) and 2-(2-fluoro-phenyl)-ethylamine (1 mL) yielding 0.28 g (74%) of (44) as pale crystals after recrystallization from ethanol. GC/EI-MS, m/z (rel. int.) 378 (M⁺, 6), 377 (8), 269 (6), 256 (100), 241 (5), 184 (6), 137 (26), 109 (9), 77 (4); RT=10.684 min.

### 3-[2-(2-Fluoro-phenyl)-ethyl]-2-(2-hydroxy-phenyl)-5-methyl-3H-quinazolin-4-one (45)

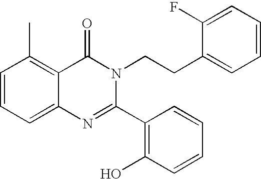

Prepared from acetic acid 2-(5-methyl-4-oxo-4H-benzo[d][1,3]oxazin-2-yl)-phenyl ester (0.30 g, 0.001 mol) and 2-(2-fluoro-phenyl)-ethylamine (1 mL) yielding 0.18 g (50%) of (45) as pale crystals after recrystallization from ethanol. GC/EI-MS, m/z (rel. int.) 374 (M⁺, 6), 373 (11), 357 (5), 265 (6), 252 (100), 237 (5), 133 (16), 109 (7), 89 (6), 77 (7); RT=11.015 min.

### 3-[2-(3-Fluoro-phenyl)-ethyl]-2-(2-hydroxy-phenyl)-5-methyl-3H-quinazolin-4-one (46)

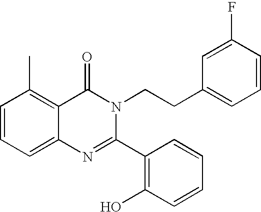

Prepared from acetic acid 2-(5-methyl-4-oxo-4H-benzo[d][1,3]oxazin-2-yl)-phenyl ester (0.30 g, 0.001 mol) and 2-(3-fluoro-phenyl)-ethylamine (1 mL) yielding 0.30 g (80%) of (46) as pale crystals after recrystallization from ethanol. GC/EI-MS, m/z (rel. int.) 374 (M⁺, 5), 373 (7), 264 (7), 252 (100), 133 (17), 109 (5), 89 (5), 77 (6); RT=10.991 min.

### 3-[2-(3-Fluoro-phenyl)-ethyl]-2-(2-hydroxy-phenyl)-6-methyl-3H-quinazolin-4-one (47)

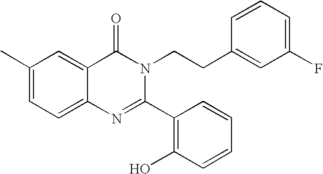

Prepared from acetic acid 2-(6-methyl-4-oxo-4H-benzo[d][1,3]oxazin-2-yl)-phenyl ester (0.30 g, 0.001 mol) and 2-(3-fluoro-phenyl)-ethylamine (1 mL) yielding 0.31 g (80%) of (47) as pale crystals after recrystallization from ethanol. GC/EI-MS, m/z (rel. int.) 374 (M⁺, 7), 373 (8), 265 (6), 252 (100), 133 (22), 104 (6), 89 (4), 77 (5); RT=11.211 min.

### 3-[2-(2-Fluoro-phenyl)-ethyl]-2-(2-hydroxy-phenyl)-6-methyl-3H-quinazolin-4-one (48)

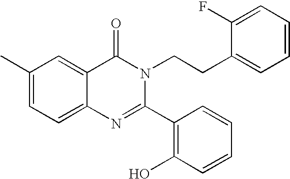

Prepared from acetic acid 2-(6-methyl-4-oxo-4H-benzo[d][1,3]oxazin-2-yl)-phenyl ester (0.30 g, 0.001 mol) and 2-(2-fluoro-phenyl)-ethylamine (1 mL) yielding 0.28 g (75%) of (48) as pale crystals after recrystallization from ethanol. GC/EI-MS, m/z (rel. int.) 374 (M⁺, 8), 373 (10), 357 (4), 265 (7), 252 (100), 237 (5), 133 (27), 104 (7), 91 (6), 77 (8); RT=11.267 min.

### 6-Chloro-3-[2-(3-fluoro-phenyl)-ethyl]-2-(2-hydroxy-phenyl)-3H-quinazolin-4-one (49)

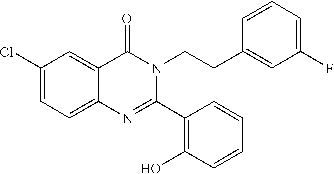

Prepared from acetic acid 2-(6-chloro-4-oxo-4H-benzo[d][1,3]oxazin-2-yl)-phenyl ester (0.32 g, 0.001 mol) and 2-(3-fluoro-phenyl)-ethylamine (1 mL) yielding 0.18 g (45%) of (49) as pale crystals after recrystallization from ethanol. GC/EI-MS, m/z (rel. int.) 395 (M⁺, 3), 394 (5), 393 (6), 284 (8), 274 (34), 272 (100), 153 (31), 109 (7), 7 (6), 75 (7); RT=11.553 min.

### 6-Chloro-3-[2-(2-fluoro-phenyl)-ethyl]-2-(2-hydroxy-phenyl)-3H-quinazolin-4-one (50)

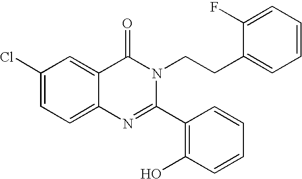

Prepared from acetic acid 2-(6-chloro-4-oxo-4H-benzo[d][1,3]oxazin-2-yl)-phenyl ester (0.32 g, 0.001 mol) and 2-(2-fluoro-phenyl)-ethylamine (1 mL) yielding 0.32 g (80%) of (50) as pale crystals after recrystallization from ethanol. GC/EI-MS, m/z (rel. int.) 395 (M⁺, 4), 394 (6), 393 (9), 285 (6), 274 (34), 272 (100), 257 (5), 152 (27), 109 (8), 77 (6); RT=11.556 min.

### 3-[2-(3-Fluoro-phenyl)-ethyl]-2-(2-hydroxy-phenyl)-6-methoxy-3H-quinazolin-4-one (51)

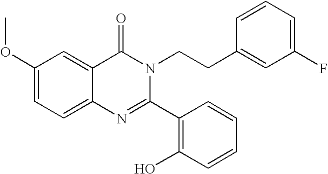

Prepared from acetic acid 2-(6-methoxy-4-oxo-4H-benzo[d][1,3]oxazin-2-yl)-phenyl ester (0.31 g, 0.001 mol) and 2-(3-fluoro-phenyl)-ethylamine (1 mL) yielding 0.31 g (80%) of (51) as pale crystals after recrystallization from ethanol. GC/EI-MS, m/z (rel. int.) 390 (M⁺, 8), 389 (8), 280 (9), 269 (18), 268 (100), 253 (6), 149 (19), 106 (12), 63 (6); RT=11.773 min.

### 3-[2-(3-Fluoro-phenyl)-ethyl]-6-hydroxy-2-(2-hydroxy-phenyl)-3H-quinazolin-4-one (52)

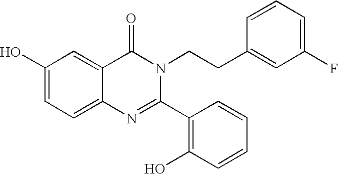

Prepared from acetic acid 2-(6-hydroxy-4-oxo-4H-benzo[d][1,3]oxazin-2-yl)-phenyl ester (0.30 g, 0.001 mol) and 2-(3-fluoro-phenyl)-ethylamine (1 mL) yielding 0.19 g (50%) of (52) as pale crystals after recrystallization from ethanol. ¹H NMR (DMSO-d₆): δ 10.70 (2H, boad s), 7.75 (1H, d, J=8.8), 7.61 (1H, d, J=2.5), 7.56-7.51 (2H, m), 7.43 (1H, dd, J₁=7.7, J₂=1.4), 7.29-7.20 (2H, m), 7.07-6.98 (2H, m), 6.69-6.61 (2H, m), 4.08 (2H, t, J=7.7), 2.87 (2H, t, J=7.4). ¹³C NMR (DMSO-d₆): δ 163.79, 160.56, 159.47, 157.83, 154.66, 153.44, 140.47, 140.37, 132.85, 130.55, 130.44, 129.89, 125.11, 124.81, 124.53, 120.85, 119.26, 118.08, 116.28, 115.27, 115.00, 113.63, 113.35, 109.76, 46.74, 33.22.

### 3-[2-(3-Fluoro-phenyl)-ethyl]-2-(2-hydroxy-phenyl)-8-methoxy-3H-quinazolin-4-one (53)

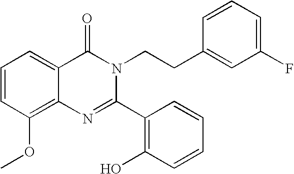

Prepared from acetic acid 2-(8-methoxy-4-oxo-4H-benzo[d][1,3]oxazin-2-yl)-phenyl ester (0.31 g, 0.001 mol) and 2-(3-fluoro-phenyl)-ethylamine (1 mL) yielding 0.2 g (50%) of (53) as pale crystals after recrystallization from ethanol. GC/EI-MS, m/z (rel. int.) 390 (M⁺, 13), 389 (16), 280 (9), 269 (18), 268 (90), 267 (56), 250 (100), 237 (30), 210*8), 132 (14), 122 (15), 91 (12) 77 (19); RT=11.593 min.

### 3-[2-(3-Fluoro-phenyl)-ethyl]-2-(2-hydroxy-phenyl)-8-trifluoromethyl-3H-quinazolin-4-one (54)

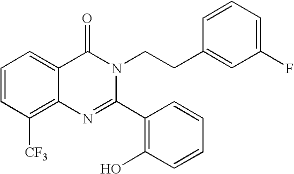

Prepared from acetic acid 2-(8-trifluoromethyl-4-oxo-4H-benzo[d][1,3]oxazin-2-yl)-phenyl ester (0.35 g, 0.001 mol) and 2-(3-fluoro-phenyl)-ethylamine (1 mL) yielding 0.25 g (58%) of (54) as pale crystals after recrystallization from ethanol. GC/EI-MS, m/z (rel. int.) 428 (M⁺, 4), 427 (6), 409 (3), 307 (18), 306 (100), 286 (31), 258 (5), 168 (12), 122 (5), 109 (4); RT=10.408 min.

### N-[3-[2-(3-Fluoro-phenyl)-ethyl]-2-(2-hydroxy-phenyl)-4-oxo-3,4-dihydro-quinazolin-6-yl]-acetamide (55)

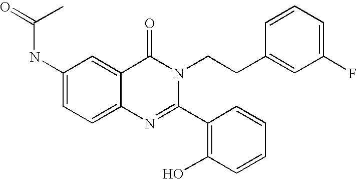

Prepared from acetic acid 2-(6-acetylamino-4-oxo-4H-benzo[d][1,3]oxazin-2-yl)-phenyl ester (0.34 g, 0.001 mol) and 2-(3-fluoro-phenyl)-ethylamine (1 mL) yielding 0.23 g (56%) of (55) as pale crystals after recrystallization from ethanol. ¹H NMR (DMSO-d₆): δ 10.35 (1H, s), 10.16 (1H, s), 8.55 (1H, s), 7.97 (1H, d, J=6.9), 7.63 (1H, d, J=8.8), 7.38 (1H, t, J=7.7), 7.33-7.18 (2H, m), 7.02-6.91 (3H, m), 6.62 (1H, d, J=7.6), 6.57 (1H, d, J=8.9), 4.04 (2H, broad t, J=7.4), 2.82 (2H, t, J=7.4), 2.12 (3H, s). ¹³C NMR (DMSO-d₆): δ 168.63, 163.77, 160.88, 160.53, 154.11, 152.76, 142.97, 140.94, 140.84, 138.05, 131.08, 130.43, 130.32, 129.88, 127.83, 125.97, 124.51, 122.76, 120.70, 119.09, 115.65, 115.22, 114.94, 114.18, 113.43, 113.15, 46.02, 33.52, 24.07.

### 8-Chloro-2-(2-hydroxy-phenyl)-3-phenethyl-3H-quinazolin-4-one (56)

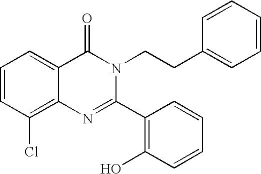

Prepared from acetic acid 2-(8-chloro-4-oxo-4H-benzo[d][1,3]oxazin-2-yl)-phenyl ester (0.32 g, 0.001 mol) and phenethylamine (1 mL) yielding 0.23 g (60%) of (56) as colorless crystals after recrystallization from ethanol. GC/EI-MS, m/z (rel. int.) 377 (M⁺, 1), 376 (2), 375 (4), 284 (7), 274 (34), 273 (20), 272 (100), 153 (34), 91 (16), 77 (14); RT=9.721 min.

### 8-Chloro-3-[2-(3-fluoro-phenyl)-ethyl]-2-(2-hydroxy-phenyl)-3H-quinazolin-4-one (57)

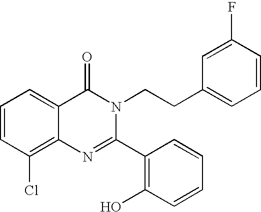

Prepared from acetic acid 2-(8-chloro-4-oxo-4H-benzo[d][1,3]oxazin-2-yl)-phenyl ester (0.32 g, 0.001 mol) and 2-(3-fluoro-phenyl)-ethylamine (1 mL) yielding 0.20 g (50%) of (57) as colorless crystals after recrystallization from ethanol. GC/EI-MS, m/z (rel. int.) 395 (M⁺, 2), 394 (3), 393 (5), 284 (10), 274 (34), 273 (20), 272 (100), 153 (40), 109 (17), 83 (11), 75 (11); RT=9.639 min.

### 7-Chloro-3-[2-(3-fluoro-phenyl)-ethyl]-2-(2-hydroxy-phenyl)-3H-quinazolin-4-one (58)

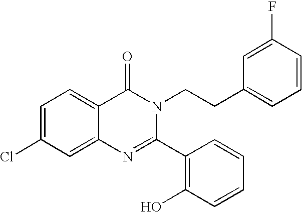

Prepared from acetic acid 2-(7-chloro-4-oxo-4H-benzo[d][1,3]oxazin-2-yl)-phenyl ester (0.32 g, 0.001 mol) and 2-(3-fluoro-phenyl)-ethylamine (1 mL) yielding 0.19 g (50%) of (58) as colorless crystals after recrystallization from ethanol. GC/EI-MS, m/z (rel. int.) 395 (M⁺, 2), 394 (3), 395 (4), 274 (34), 273 (22), 271 (100), 153 (36), 122 (49), 109 (59), 83 (30), 75 (17); RT=9.608 min.

### 7-Chloro-3-[2-(2-fluoro-phenyl)-ethyl]-2-(2-hydroxy-phenyl)-3H-quinazolin-4-one (59)

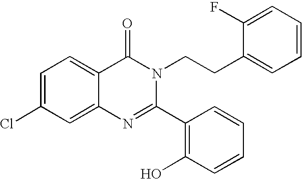

Prepared from acetic acid 2-(7-chloro-4-oxo-4H-benzo[d][1,3]oxazin-2-yl)-phenyl ester (0.32 g, 0.001 mol) and 2-(2-fluoro-phenyl)-ethylamine (1 mL) yielding 0.20 g (50%) of (59) as colorless crystals after recrystallization from ethanol. GC/EI-MS, m/z (rel. int.) 395 (M⁺, 3), 394 (4), 393 (7), 285 (6), 274 (34), 273 (20), 272 (100), 153 (20), 109 (20), 91 (7), 75 (11); RT=9.649 min.

### 8-Chloro-3-[2-(2-fluoro-phenyl)-ethyl]-2-(2-hydroxy-phenyl)-3H-quinazolin-4-one (60)

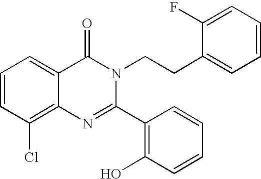

Prepared from acetic acid 2-(8-chloro-4-oxo-4H-benzo[d][1,3]oxazin-2-yl)-phenyl ester (0.32 g, 0.001 mol) and 2-(2-fluoro-phenyl)-ethylamine (1 mL). The final mixture was poured onto ice water yielding 0.23 g (60%) of (60) as light yellow crystals after recrystallization from ethanol. GC/EI-MS, m/z (rel. int.) 395 (M⁺, 3), 394 (5), 393 (8), 284(7), 274 (35), 273 (20), 272 (100), 153 (35), 109 (12), 103 (8), 77 (9); RT=9.665 min.

### 2-(2-Hydroxy-phenyl)-3-(2-pyridin-3-yl-ethyl)-3H-quinazolin-4-one (61)

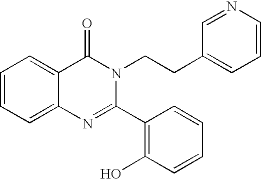

Prepared from acetic acid 2-(4-oxo-4H-benzo[d][1,3]oxazin-2-yl)-phenyl ester (0.30 g, 0.001 mol) and 2-pyridin-3-yl-ethylamine (1 mL). The final mixture was poured onto ice water yielding 0.22 g (65%) of (61) as light yellow crystals after recrystallization from ethanol. GC/EI-MS, m/z (rel. int.) 343 (M⁺, 8), 342 (10), 326 (6), 250 (17), 239 (34), 238 (100), 166 (18), 105 (34), 92 (52), 77 (34), 65 (50), 51)18); RT=9.556 min.

### 6-Fluoro-2-(2-hydroxy-phenyl)-3-(2-pyridin-3-yl-ethyl)-3H-quinazolin-4-one (62)

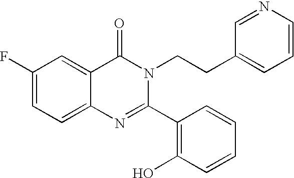

Prepared from acetic acid 2-(6-fluoro-4-oxo-4H-benzo[d][1,3]oxazin-2-yl)-phenyl ester (0.32 g, 0.001 mol) and 2-pyridin-3-yl-ethylamine (1 mL). The final mixture was poured onto ice water yielding 0.23 g (60%) of (62) as light yellow crystals after recrystallization from ethanol. GC/EI-MS, m/z (rel. int.) 361 (M⁺, 13), 360 (15), 344 (10), 268 (18), 257 (27), 256 (100), 184 (24), 137 (60), 106 (50), 94 (21), 65 (36), 51 (14); RT=9.469 min.

### 3-Phenethyl-2-pyridin-4-yl-3H-quinazolin-4-one (63)

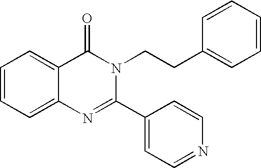

Prepared from 2-pyridin-4-yl-benzo[d][1,3]oxazin-4-one (0.22 g, 0.001 mol) and phenethylamine (1 mL). The final mixture was poured onto ice water yielding 0.16 g (50%) of (63) as colorless crystals after recrystallization from ethanol. GC/EI-MS, m/z (rel. int.) 328 (M⁺+1, 17), 327 (M⁺, 73), 236 (36), 224 (27), 223 (90), 207 (12), 119 (43), 104 (100), 77 (27).

### 6-Fluoro-2-(2-hydroxy-phenyl)-3-(3-phenyl-propyl)-3H-quinazolin-4-one (64)

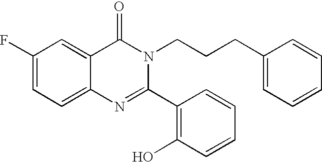

Prepared from acetic acid 2-(6-fluoro-4-oxo-4H-benzo[d][1,3]oxazin-2-yl)-phenyl ester (0.30 g, 0.001 mol) and 3-phenyl-propylamine (1 mL) yielding 0.26 g (70%) of (64) as colorless after recrystallization from ethanol. GC/EI-MS, m/z (rel. int.) 374 (M⁺, 27), 373 (23), 357 (6), 283(25), 269 (100), 257 (30), 256 (54), 137 (23), 01 (24), 65 (6); RT=11.534 min.

### 6-Fluoro-2-(2-hydroxy-phenyl)-3-(1-methyl-1-pyridin-3-yl-ethyl)-3H-quinazolin-4-one (65)

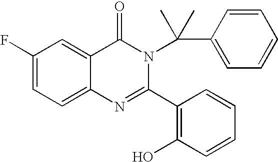

Prepared from acetic acid 2-(6-fluoro-4-oxo-4H-benzo[d][1,3]oxazin-2-yl)-phenyl ester (0.30 g, 0.001 mol) and 1-methyl-1-phenyl-ethylamine (1 mL) yielding 0.26 g (70%) of (65) as colorless after recrystallization from ethanol. ¹H NMR (DMSO-d₆) (mixture of atropoisomers): δ 11.52 (1H, OH, s), 11.30 (1H, OH, s), 8.78 (1H, s), 8.27 (1H, dd, J=7.3, J₂=5.3), 7.82 (1H, dd, J=4.8, J₂=1.7), 7.62 (1H, dd, J₁=6.0, J₂=3.0), 7.43-7.35 (4H, m), 7.22-7.12 (3H, m), 6.97-6.90 (2H, m), 1.63 (6H, s). ¹³C NMR (DMSO-d₆): δ 166.03, 164.98, 159.21, 157.63, 156.0, 147.18, 133.63, 132.98, 132.94, 129.76, 128.75, 128.67, 128.87, 125.80, 124.91, 124.82, 124.63, 119.19, 117.78, 117.30, 116.98, 115.32, 115.0, 55.89, 29.35.

### 3-(1,1-Dimethyl-2-phenyl-ethyl)-6-fluoro-2-(2-hydroxy-phenyl)-3H-quinazolin-4-one (66)

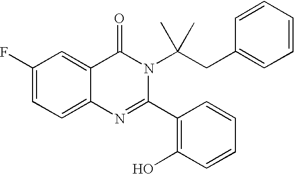

Prepared from acetic acid 2-(6-fluoro-4-oxo-4H-benzo[d][1,3]oxazin-2-yl)-phenyl ester (0.30 g, 0.001 mol) and 1,1-dimethyl-3-phenyl-ethyllamine (1 mL) yielding 0.30 g (77%) of (66) as colorless after recrystallization from ethanol. ¹H NMR (DMSO-d₆) (mixture of atropoisomers): δ 11.63 (1H, OH, s), 11.52 (1H, OH, s), 8.27 (1H, dd, J₁=7.3, J₂=5.3), 8.0 (1H, dd, J=4.9, J₂=1.8), 7.95 (1H, s), 7.44-7.30 (2H, m), 7.20-6.96 (7H, m), 3.12 (2H, s), 1.34 (6H, s). ¹³C NMR (DMSO-d-₆): δ 166.79, 164.0, 158.91, 157.20, 155.70, 138.13, 133.59, 132.90, 132.86, 130.32, 129.16, 129.08, 127.61, 126.03, 125.03, 124.93, 119.34, 118.21, 116.93, 116.71, 115.19, 114.87, 54.25, 43.28, 26.91.

### 3-(1,1-Dimethyl-3-phenyl-propyl)-6-fluoro-2-(2-hydroxy-phenyl)-3H-quinazolin-4-one (67)

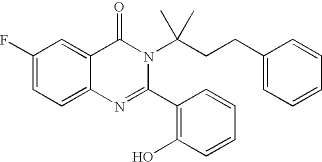

Prepared from acetic acid 2-(6-fluoro-4-oxo-4H-benzo[d][1,3]oxazin-2-yl)-phenyl ester (0.30 g, 0.001 mol) and 1,1-dimethyl-3-phenyl-propylamine (1 mL) yielding 0.28 g (70%) of (67) as colorless after recrystallization from ethanol. ¹H NMR (DMSO-d₆) (mixture of atropoisomers): δ 11.62 (1H, OH, s), 11.52 (1H, OH, s), 8.29 (1H, dd, J₁=7.1, J₂=5.3), 8.16 (1H, s), 7.95 (1H, dd, J₁=4.8, J₂=1.7), 7.48-7.33 (3H, m), 7.07-6.94 (7H, m), 2.52-2.47 (2H, m), 2.08-2.02 (2H, m), 1.39 (6H, s).

### 6-Fluoro-2-(2-hydroxy-phenyl)-3-(2-phenyl-propyl)-3H-quinazolin-4-one (68)

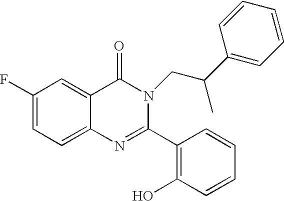

Prepared from acetic acid 2-(6-fluoro-4-oxo-4H-benzo[d][1,3]oxazin-2-yl)-phenyl ester (0.32 g, 0.001 mol) and 2-phenyl-propylamine (1 mL) yielding 0.23 g (57%) of (68) as colorless crystals after purification by MPLC using a gradient of ethyl acetate-hexanes (100% hexanes; 10% ethyl acetate-hexanes; 20% ethyl acetate-hexanes; finally, 30% ethyl acetate-hexanes). ¹H NMR (DMSO-d₆): δ 9.60 (1H, OH, s), 7.69 (1H, dd, J₁=5.6, J₂=2.7), 7.49-7.33 (2H, m), 7.20-7.38 (3H, m), 6.92-6.85 (2H, m), 6.72-6.67 (3H, m), 4.46-4.23 (1H, m), 4.10-4.05 (1H, m), 3.18-3.02 (1H, m), 1.06 (3H, d, J=6.9).

### 6-Fluoro-2-(2-hydroxy-phenyl)-3-(R-2-phenyl-propyl)-3H-quinazolin-4-one (69)

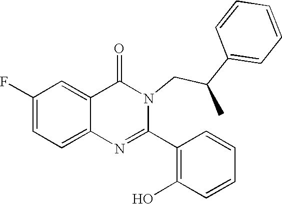

Prepared from acetic acid 2-(6-fluoro-4-oxo-4H-benzo[d][1,3]oxazin-2-yl)-phenyl ester (0.32 g, 0.001 mol) and R-2-phenyl-propylamine (1 mL) yielding 0.34 g (87%) of (69) as colorless crystals after purification by MPLC using a gradient of ethyl acetate-hexanes (100% hexanes; 10% ethyl acetate-hexanes; 20% ethyl acetate-hexanes; 30% ethyl acetate-hexanes; finally, 40% ethyl acetate-hexanes). ¹H NMR (DMSO-d₆): δ 9.70 (1H, OH, s), 7.68 (1H, dd, J₁=5.6, J₂=2.7), 7.47-7.34 (2H, m), 7.26-7.03 (4H, m), 6.82 (2H, d, J=4.7), 6.69-6.64 (3H, m), 4.45-4.36 (1H, m), 4.02-3.95 (1H, m), 3.09-2.88 (1H, m), 1.07 (3H, d, J=7.1).

### 6-Fluoro-2-(2-hydroxy-phenyl)-3-(S-2-phenyl-propyl)-3H-quinazolin-4-one (70)

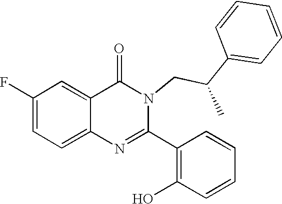

Prepared from acetic acid 2-(6-fluoro-4-oxo-4H-benzo[d][1,3]oxazin-2-yl)-phenyl ester (0.32 g, 0.001 mol) and S-2-phenyl-propylamine (1 mL) yielding 0.32 g (72%) of (70) as colorless crystals after purification by MPLC using a gradient of ethyl acetate-hexanes (100% hexanes; 10% ethyl acetate-hexanes; 20% ethyl acetate-hexanes; finally, 30% ethyl acetate-hexanes). ¹H NMR (DMSO-d₆): δ 9.60 (1H, OH, s), 7.68 (1H, dd, J₁=5.5, J₂=2.5), 7.47-7.35 (2H, m), 7.20-7.03 (4H, m), 6.84 (2H, d, J=4.9), 6.89-6.62 (3H, m), 4.46-4.38 (1H, m), 4.08-3.98 (1H, m), 3.09-2.86 (1H, m), 1.07 (3H, d, J=6.8).

### 3-(2,2-Diphenyl-ethyl)-6-fluoro-2-(2-hydroxy-phenyl)-3H-quinazolin-4-one (71)

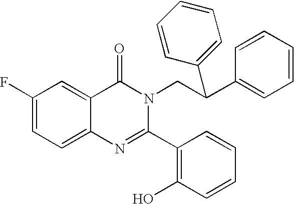

Prepared from acetic acid 2-(6-fluoro-4-oxo-4H-benzo[d][1,3]oxazin-2-yl)-phenyl ester (0.32 g, 0.001 mol) and 2,2-diphenyl-ethylamine (1 mL) yielding 0.39 g (81%) of (71) as colorless crystals after purification by MPLC using a gradient of ethyl acetate-hexanes (100% hexanes; 10% ethyl acetate-hexanes; 20% ethyl acetate-hexanes; 30% ethyl acetate-hexanes; finally, 40% ethyl acetate-hexanes). ¹H NMR (DMSO-d₆): δ 9.50 (1H, OH, s), 7.70 (1H, dd, J₁=5.5, J₂=2.5), 7.45-7.33 (3H, m), 7.27-7.21 (2H, m), 7.13-7.10 (6H, m), 6.91-6.78 (5H, m), 4.82 (2H, d, J=7.4), 4.35 (1H, d, J=7.8).

### EXAMPLE 3

### Preparation of 2-(2-Hydroxy-phenyl)-3-phenethyl-3H-pyrido[2,3-d]pyrimidin-4-one (73)

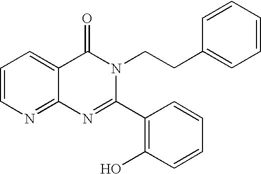

A mixture of acetic acid 2-(2-hydroxy-phenyl)-pyrido[2,3-d][1,3]oxazin-4-one ester (0.28 g, 0.001 mol), phenethylamine (1 mL) and pyridine (6 mL) was refluxed for 2 h, cooled and poured onto ice water (25 mL). The colorless precipitate was filtered off and recrystallized from ethanol to give 0.24 g (70%) of (73) as colorless crystals. GC/EI-MS, m/z (rel. int.) 343 (M⁺, 2), 342 (7), 239 (20), 239 (100), 211 (12), 91 (12), 65 (6); RT=9.792 min.

In a similar manner, the following pyridopyrimidin-4-one was prepared:

### 3-[2-(3-Fluoro-phenyl)-ethyl]-2-(2-hydroxy-phenyl)-3H-pyrido[2,3-d]pyrimidin-4-one (74)

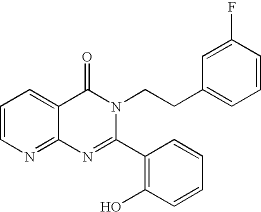

Prepared from acetic acid 2-(2-hydroxy-phenyl)-pyrido[2,3-d][1,3]oxazin-4-one ester (0.28 g, 0.001 mol) and 2-(3-flouro-phenyl)-ethylamine (1 mL) yielding 0.27 g (75%) of (74) as colorless crystals from ethanol. ¹H NMR (DMSO-d₆): δ 10.26 (1H, OH, s), 9.03-9.01 (1H, m), 7.62 (1H, dd, J₁=6.2, J₂=4.5), 7.44 (1H, t, J=7.8), 7.27-7.20 (2H, m), 7.06-6.95 (3H, m), 6.64-6.57 (2H, m), 4.08 (2H, t, J=7.4), 2.85 (2H, t, J=7.4). ¹³C NMR (DMSO-d₆): δ 163.77, 161.57, 160.54, 157.54, 156.99, 156.16, 153.83, 140.74, 140.64, 135.95, 131.39, 130.45, 130.35, 129.59, 124.51, 122.77, 122.42, 119.18, 115.77, 115.52, 115.25, 114.97, 113.48, 113.21, 46.28, 33.36.

### EXAMPLE 4

### Preparation of 2-(2-Hydroxy-phenyl)-3-phenethyl-5-phenethylamino-3H-quinazolin-4-one (75)

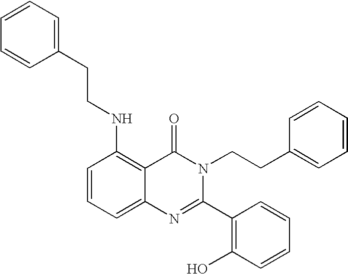

Prepared from acetic acid 2-(5-chloro-4-oxo-4H-benzo[d][1,3]oxazin-2-yl)-phenyl ester (0.32 g, 0.001 mol) and phenethylamine (1 mL) yielding 0.23 g (50%) of (75), or from acetic acid 2-(5-fluoro-4-oxo-4H-benzo[d][1,3]oxazin-2-yl)-phenyl ester (0.30 g, 0.001 mol) and phenethylamine (1 mL) yielding 0.3 g (65%) of (75) as colorless crystals after recrystallization from ethanol. ¹H NMR (DMSO-d₆): δ 11.10 (1H, broad s), 7.68 (1H, t, J=8.2), 7.57-7.52 (1H, m), 7.43 (1H, dd, J=7.4, 1.4), 7.37-7.34 (4H, m), 7.27-7.20 (5H, m), 7.05 (1H, t, J=7.4), 6.91-6.81 (4H, m), 3.97 (2H, t, J=6.9), 3.57 (2H, t, J=7.1), 2.98 (2H, t, J=7.4), 2.82 (2H, t, J=7.7). ¹³C NMR (DMSO-d₆): δ 161.56, 156.40, 154.58, 150.08, 139.03, 137.42, 136.85, 133.13, 129.59, 128.79, 128.63, 128.40, 128.30, 126.66, 126.30, 119.21, 117.15, 116.35, 108.11, 106.683, 102.96, 46.84, 43.88, 34.34, 33.52.

In a similar manner, the following quinazolin-4-ones were prepared:

### 3-[2-(3-Fluoro-phenyl)-ethyl]-7-[2-(3-fluoro-phenyl)-ethylamino]-2-(2-hydroxy-phenyl)-3H-quinazolin-4-one (76)

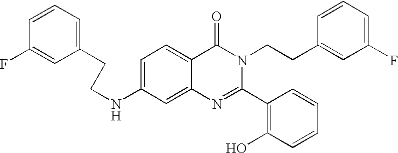

Prepared from acetic acid 2-(7-fluoro-4-oxo-4H-benzo[d][1,3]oxazin-2-yl)-phenyl ester (0.32 g, 0.001 mol) and 2-(3-flouro-phenyl)-ethylamine (1 mL) yielding 0.25 g (50%) of (76) as colorless crystals after recrystallization from ethanol. ¹H NMR (DMSO-d₆): δ 11.18 (1H, OH, broad s), 7.95 (1H, d, J=9.0), 7.57 (1H, t, J=8.0), 7.48 (1H, d, J=7.7), 7.39-7.16 (6H, m), 7.09-6.99 (4H, m), 6.84 (1H, s), 6.70-6.30 (2H, m), 4.04 (2H, broad s), 3.42 (2H, t, J=7.3), 2.95-2.84 (4H, m). ¹³C NMR (DMSO-d₆): δ 163.83, 163.78, 160.61, 160.54, 158.04, 157.18, 154.54, 142.12, 142.02, 140.34, 140.24, 133.45, 130.56, 130.45, 130.24, 130.12, 129.58, 128.24, 124.96, 124.92, 124.50, 119.28, 116.41, 115.64, 115.37, 115.26, 114.98, 113.66, 113.38, 113.15, 112.88, 107.01, 100.10, 46.58, 43.60, 33.67, 33.31.

### 3-Phenethyl-2-(2-phenethylamino-phenyl)-3H-quinazolin-4-one (77)

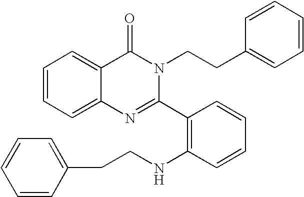

Prepared from 2-(2-fluoro-phenyl)-benzo[d][1,3]oxazin-4-one (0.24 g, 0.001 mol) and phenethylamine (1 mL) yielding 0.22 g (50%) of (77) as colorless crystals from ethanol. GC/EI-MS, m/z (rel. int.) 445 (M⁺, 4), 354 (100), 340 (5), 262 (4), 250 (59), 233 (13), 119 (13), 104 (44), 91 (22), 77 (15), 51 (2); RT=14.842 min.

### EXAMPLE 5

### Preparation of 2-Methyl-3H-quinazolin-4-one (79)

### 2-Methyl-benzo[d][1,3]oxazin-4-one (78)

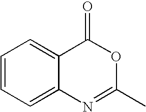

A mixture of anthranilic acid (100 g, 0.73 mol) and acetic anhydride (300 mL) was refluxed for 1 h, and excess of acetic anhydride was removed in vacuum. The residue was cooled and treated with diethyl ether to give bulky precipitate, which was filtered off, washed with cold ether and dried in vacuum overnight at room temperature to give 105 g (90%) of (78) as colorless needles. GC/EI-MS, m/z (rel. int.) 161 (M⁺, 89), 146 (100), 117 (63), 90 (54), 76 (18), 63 (21), 50 (28), 43 (45); RT=4.013 min.

### 2-Methyl-3H-quinazolin-4-one (79)

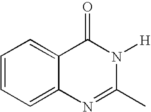

A mixture of 2-methyl-benzo[d][1,3]oxazin-4-one (78) (0.32 g, 0.002 mol) and aqueous ammonium hydroxide (6 mL) was stirred at room temperature for 18 h. The residue was filtered off, washed with water and dried. Recrystallization from ethanol yielded 0.2 g (62%) of (79) as colorless needles. GC/EI-MS, m/z (rel. int.) 160 (M⁺, 100), 159 (12), 145 (13), 131 (10), 118 (23), 90 (27), 76 (13), 63 (17), 50 (14), 42 (25); RT=10.406 min.

In a similar manner, the following quinazolin-4-ones were prepared:

### 2,3-Dimethyl-3H-quinazolin-4-one (80)

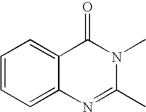

Prepared from 2-methyl-benzo[d][1,3]oxazin-4-one (3.2 g, 0.02 mol) and aqueous solution of methyl amine (20 mL) yielding 3.2 g (94%) of (80) as colorless needles. GC/EI-MS, m/z (rel. int.) 174 (M⁺, 100), 159 (100), 146 (100), 313 (42), 117 (39), 89 (46), 76 (49), 56 (100), 49 (36), 41 (9); RT=10.722 min.

### 3-Methyl-2-phenethyl-3H-quinazolin-4-one (81)

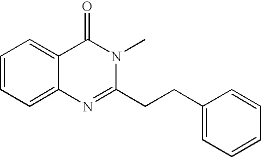

Prepared from 2-phenethyl-benzo[d][1,3]oxazin-4-one (0.25 g, 0.001 mol) and aqueous solution of methyl amine (5 mL) yielding 0.26 g (100%) of (81) as colorless needles. GC/EI-MS, m/z (rel. int.) 264 (M⁺, 100), 233 (2), 187 (25), 160 (29), 131 (12), 119 (26), 91 (26), 77 (11), 51 (4); RT=9.825 min.

### EXAMPLE 4

### Preparation of 3-Methyl-2-styryl-3H-quinazolin-4-one (82)

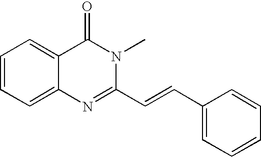

A mixture of 2,3-dimethyl-3H-quinazolin-4-one (0.17 g, 0.001 mol), benzaldehyde (0.11 g, 0.001 mol) and catalytic amount of piperidine was fused at 175° C. for 30 min. The residue was cooled and treated with hexane. The yellow solid was filtered off and recrystallizied from ethanol to give 0.16 g (60%) of (82) as yellow needles. GC/EI-MS, m/z (rel. int.) 261 [(M⁺−1), 100], 247 (10), 2233 (17), 218 (7), 204 (12), 185 (73), 128 (12), 115 (14), 102 (25), 90 (18), 77 (29), 63 (14), 50 (15), 42 (2); RT=9.894 min.

In a similar manner, the following quinazolin-4-ones were prepared:

### 3-Methyl-2-(2-p-tolyl-vinyl)-3H-quinazolin-4-one (83)

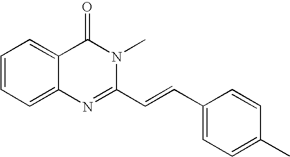

Prepared from 2,3-dimethyl-3H-quinazolin-4-one (0.17 g, 0.001 mol) and p-tolualdehyde (0.12 g, 0.001 mol) yielding 0.12 g (44%) of (83) as yellow crystals. GC/EI-MS, m/z (rel. int.) 276 (M⁺, 100), 261 (29), 247 (29), 313 (14), 217 (13), 185 (100), 157 (8), 142 (15), 130 (14), 115 (47), 90 (26), 76 (16), 63 (15), 50 (11); RT=10.406 min.

### 2-[2-(4-Chloro-phenyl)-vinyl]-3-methyl-3H-quinazolin-4-one (84)

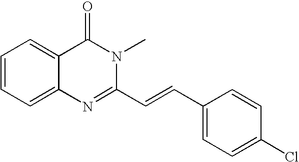

Prepared from 2,3-dimethyl-3H-quinazolin-4-one (0.17 g, 0.001 mol) and 4-chlorobenzaldehyde (0.14 g, 0.001 mol) yielding 0.24 g (83%) of (84) as yellow crystals. GC/EI-MS, m/z (rel. int.) 296 (M⁺, 67), 295 (100), 281 (5); 267 (10), 203 (7), 185 (75), 128 (14), 119 (9), 101 (22), 90 (17), 75 (18), 63 (12), 50 (12); RT=10.722 min.

### 2-[2-(4-Fluoro-phenyl)-vinyl]-3-methyl-3H-quinazolin-4-one (85)

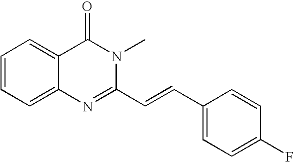

Prepared from 2,3-dimethyl-3H-quinazolin-4-one (0.17 g, 0.001 mol) and 4-fluorobenzaldehyde (0.12 g, 0.001 mol) yielding 0.18 g (64%) of (85) as yellow crystals. GC/EI-MS, m/z (rel. int.) 279 [(M⁺−1), 100], 265 (6), 251 (11), 222 (8), 185 (54), 146 (9), 120 (17), 101 (13), 90 (13), 75 (12), 63 (9), 50 (8); RT=9.825 min.

### 3-Methyl-2-(2-m-tolyl-vinyl)-3H-quinazolin-4-one (86)

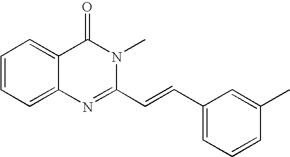

Prepared from 2,3-dimethyl-3H-quinazolin-4-one (0.17 g, 0.001 mol) and m-tolualdehyde (0.12 g, 0.001 mol) yielding 0.2 g (74%) of (86) as yellow crystals. GC/EI-MS, m/z (rel. int.) 275 [(M⁺−1), 100], 261 (10), 247 (7), 185 (56), 115 (12), 90 (6), 75 (8), 63 (5), 50 (3); RT=10.283 min.

### 3-Methyl-2-(2-o-tolyl-vinyl)-3H-quinazolin-4-one (87)

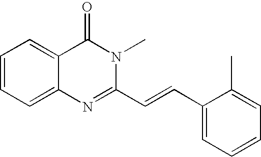

Prepared from 2,3-dimethyl-3H-quinazolin-4-one (0.17 g, 0.001 mol) and m-tolualdehyde (0.12 g, 0.001 mol) yielding 0.14 g (52%) of (87) as yellow crystals. GC/EI-MS, m/z (rel. int.) 275 [(M⁺−1), 88], 261 (100), 247 (17), 185 (67), 160 (27), 157 (16), 131 (19), 119 (34), 115 (74), 90 (29), 76 (20) 63 (17), 50 (10); RT=10.101 min.

### 3-Phenethyl-2-styryl-3H-quinazolin-4-one (88)

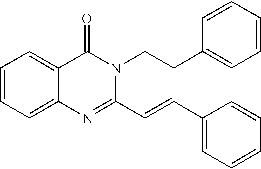

Prepared from 2-methyl-3-phenethyl-3H-quinazolin-4-one, 0.26 g (0.001 mol) and benzaldehyde (0.10 g, 0.001 mol) yielding 0.17 g (50%) of (88) as pale yellow crystals. GC/EI-MS, m/z (rel. int.) 352 (M⁺, 14), 261 (11), 247 (100), 204 (8), 103 (7), 77 (8), 65 (3), 51 (2); RT=12.097 min.

### 2-[2-(2-Fluoro-phenyl)-vinyl]-3-methyl-3H-quinazolin-4-one (89)

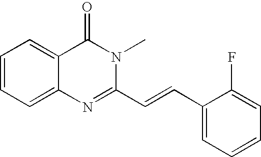

Prepared from 2,3-dimethyl-3H-quinazolin-4-one (0.17 g, 0.001 mol) and 2-fluorobenzaldehyde (0.12 g, 0.001 mol) yielding 0.15 g (54%) of (89) as yellow crystals. GC/EI-MS, m/z (rel. int.) 280 (M⁺, 59), 279 (100), 261 (31), 185 (50), 146 (10), 119 (8), 101(8), 90 (6), 75 (5), 50 (2); RT=9.770 min.

### 2-[2-(3-Fluoro-phenyl)-vinyl]-3-methyl-3H-quinazolin-4-one (90)

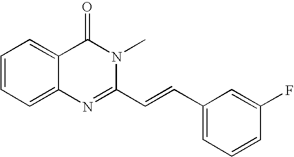

Prepared from 2,3-dimethyl-3H-quinazolin-4-one (0.17 g, 0.001 mol) and 3-fluorobenzaldehyde (0.12 g, 0.001 mol) yielding 0.15 g (54%) of (90) as yellow crystals. GC/EI-MS, m/z (rel. int.) 280 (M⁺, 63), 279 (100), 251 (11), 222 (7), 185 (56), 120 (9), 101 (7), 90 (7), 75 (6), 63 (4), 50 (3); RT=9.756 min.

### EXAMPLE 5

### Preparation of 3-Phenylethylquinazolin-4-[3H]-one (92)

### 2-Amino-N-phenethyl-benzamide (91)

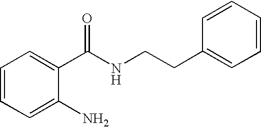

A mixture of isatoic anhydride (16.3 g, 0.1 mol) and phenylethylamine (13.3 g, 0.11 mol) was heated at 120° C. for 4 h, cooled and treated with methanol (50 mL). The colorless precipitate was filtered off and dried to give 14.4 g (60%) of pure (91). GC/EI-MS, m/z (rel. int.) 240 (M⁺, 12), 136 (24), 120 (100), 92 (26), 77 (3), 65 (23); RT=8.453 min.

### 3-Phenethyl-3H-quinazolin-4-one (92)

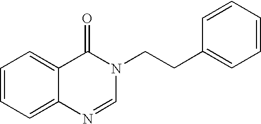

A mixture of 2-amino-N-phenethyl-benzamide (1.2 g, 0.005 mol) and orthoformate (6 mL) was refluxed for 6 h. Orthoformate was removed in vacuum, and a sirup sesidue was treated with hexane. The precipitate was filtered off and recrystallized from ethanol to give 0.76 g (76%) of (92) as colorless crystals. GC/EI-MS, m/z (rel. int.) 250 (M⁺, 16), 146 (11), 129 (31), 120 (8), 104 (100), 91 (23), 77 (28), 65 (12), 50 (9), 41 (4); RT=8,393 min.

### EXAMPLE 6

### Preparation of 2-(2-Fluoro-phenyl)-3-phenethyl-3H-quinazolin-4-one (94)

### 2-(2-Fluoro-benzoyl)amino-N-(2-phenylethyl)-benzamide (93)

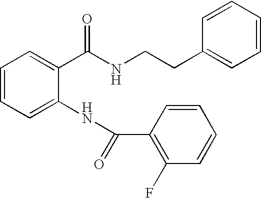

A mixture of 2-(2-fluoro-phenyl)-benzo[d][1,3]oxazin-4-one (0.24 g, 0.001 mol), 2-(3-fluoro-phenyl)-ethylamine (1 mL) and pyridine (6 mL) was refluxed for 4 h, cooled and poured onto ice with 106% HCl (6 mL). The residue was filtered off and recrystallized from ethanol to give 0.22 g (60%) of (93) as colorless crystals. GC/EI-MS, m/z (rel. int.) 362 (M⁺, 3), 258 (20), 242 (100), 214 (13), 146 (10), 123 (54), 95 (17), 65 (4); RT=11.704 min.

### 2-(2-Fluoro-phenyl)-3-phenethyl-3H-quinazolin-4-one (94)

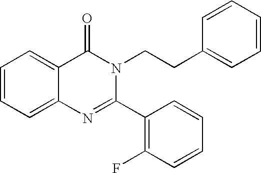

Neat 2-(2-fluoro-benzoyl)amino-N-(2-phenylethyl)-benzamide (0.164 g, 0.0005 mol) was heated at 200° C. for 48 h. The residue was purified by column chromatography on silica gel (eluent CHCl₃-EtOAc, 15:1, R_{f} 0.7) to give 0.09 g (50%) of (94) as pale yellow crystals. GC/EI-MS, m/z (rel. int.) 344 (M⁺, 9), 252 (15), 240 (100), 223 (10), 119 (21), 104 (7), 77 (8); RT=10.347 min.

In a similar manner, the following quinazolin-4-ones were prepared:

### 2-(2-Methoxy-phenyl)-3-phenethyl-3H-quinazolin-4-one (95)

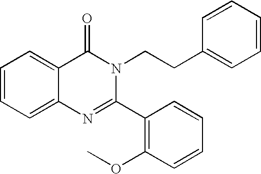

Prepared from 2-(2-methoxy-benzoyl)amino-N-(2-phenylethyl)-benzamide [¹H NMR (CDCl₃): δ 11.66 (1H, NH, s), 8.62 (1H, d, J=8.2), 8.18 (1H, dd, J=8.0, 1.9), 7.49-6.91 (12H, m), 6.46 (1H, NH, t, J=5.2), 4.07 (3H, s), 3.69-3.62 (2H, m), 2.91 (2H, t, J=6.9). ¹³C NMR (CDCl₃): δ 168.70, 164.16, 157.65, 138.65, 138.05, 133.06, 132.15, 131.35, 128.65, 128.59, 126.59, 126.51, 124.42, 122.95, 122.86, 122.25, 120.81, 111.30, 55.57, 40.96, 35.55. GC/EI-MS, m/z (rel. int.) 374 (M⁺, 5), 254 (95), 235 (32), 135 (100), 119 (33), 92 (31), 77 (45), 51 (7); RT=12.900 min.] (1.265 g, 0.005 mol) to give 0.35 g (20%) of (95) as colorless crystals after purification by column chromatography on silica gel (eluent CHCl₃—EtOAc, 10:1, R_{f} 0.75). ¹H NMR (CDCl₃): δ 8.37 (1H, d, J=8.0), 7.76-7.74 (2H, m), 7.54-7.47 (2H, m), 7.22-7.15 (4H, m), 7.10-7.05 (1H, m), 6.99 (1H, d, J=8.5), 6.86-6.83 (2H, m), 4.48-4.39 (1H, m), 3.77 (3H, s), 3.75-3.67 (1H, m), 2.98-2.89 (1H, m), 2.86-2.76 (1H, m). ¹³C NMR (CDCl₃): δ 161.86, 155.97, 154.11, 147.45, 137.98, 134.04, 131.33, 129.59, 128.64, 128.42, 127.44, 126.79, 126.57, 126.37, 124.43, 121.03, 120.99, 110.83, 55.39, 47.19, 34.51. GC/EI-MS, m/z (rel. int.) 356 (M⁺, 4), 325 (2), 265 (9), 252 (7), 234 (100), 222 (9), 179 (3), 119 (12), 77 (9), 51 (2); RT=11.076 min.

### 3-Butyl-6-fluoro-2-(2-hydroxy-phenyl)-3H-quinazolin-4-one (96)

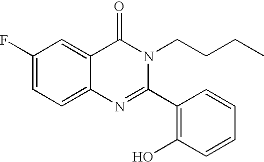

Prepared from 5-fluoro-2-(2-hydroxy-benzoyl)amino-N-(2-butyl)-benzamide (0.17 g, 0.0005 mol) on heating at 230° C. for 4 h. Yield 0.12 g (80%) of (96) as colorless crystals from ethanol. GC/EI-MS, m/z (rel. int.) 312 (M⁺, 41), 311 (100), 295 (33), 269 (65), 257 (45), 256 (91), 184 (12), 137 (21).

### 6-Fluoro-2-(2-hydroxy-phenyl)-3-propyl-3H-quinazolin-4-one (97)

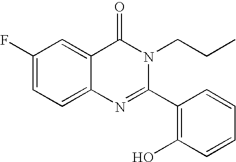

Prepared from 5-fluoro-2-(2-hydroxy-benzoyl)amino-N-(2-propyl)-benzamide (0.17 g, 0.0005 mol) on heating at 230° C. for 4 h. Yield 0.13 g (87%) of (97) as colorless crystals from ethanol. GC/EI-MS, m/z (rel. int.) 298 (M⁺, 36), 297 (100), 281 (39), 256 (71), 137 (54), 109 (18), 91 (14), 41 (20).

### 6-Fluoro-2-(2-hydroxy-phenyl)-3-(3-methyl-butyl)-3H-quinazolin-4-one (98)

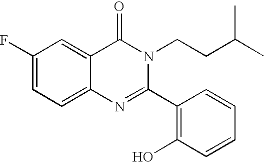

Prepared from 5-fluoro-2-(2-hydroxy-benzoyl)amino-N-(3-methyl-butyl)-benzamide (0.17 g, 0.0005 mol) on heating at 230° C. for 4 h. Yield 0.12 g (80%) of (98) as colorless crystals from ethanol. GC/EI-MS, m/z (rel. int.) 326 (M⁺, 26), 325 (42, 309 (11), 283 (35), 270 (25), 269 (100), 257 (50), 256 (89), 137 (46), 109 (11), 41 (11).

### EXAMPLE 7

### Preparation of 3-Phenethyl-2-phenyl-2,3-dihydro-1H-quinazolin-4-one (99)

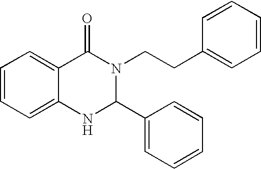

A mixture of 2-amino-N-phenethylbenzamide (91) (0.24 g, 0.001 mol) and benzaldehyde (0.159 g, 0.0015 mol) in ethanol (6 mL) was refluxed for 6 h. The solvent was removed and the residue was recrystallized from ethanol to give 0.28 g (74%) of (99) as colorless crystals. GC/EI-MS, m/z (rel. int.) 328 (M⁺, 5), 327 (5), 251 (30), 223 (39), 208 (100), 180 (10), 147 (55), 119 (14), 105 (27), 91 (39), 77 (39), 65 (19), 51 (14); RT=11.297 min.

### EXAMPLE 8

### Preparation of acetic acid 2-{6-fluoro-3-[2-(3-fluoro-phenyl)-ethyl]-4-oxo-3,4-dihydro-quinazolin-2-yl}-phenyl ester (100)

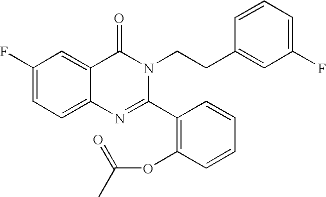

A mixture of 6-fluoro-3-[2-(3-fluoro-phenyl)-ethyl]-2-(2-hydroxy-phenyl)-3H-quinazolin-4-one (0.3 g, 0.001 mol) and acetic anhydride was refluxed for 4 h. The excess of acetic anhydride was removed in vacuo, the residue was treated with diethyl ether (10 mL), and the product was filtered off and recrystallized from ethanol to give 0.32 g (79%) of (100) as colorless crystals. GC/EI-MS, m/z (rel. int.) 420 (M⁺, 1), 377 (4), 298 (6), 269 (7), 256 (100), 137 (13), 43 (9).

### EXAMPLE 9

### Preparation of isobutyric acid 2-{6-fluoro-3-[2-(3-fluoro-phenyl)-ethyl]-4-oxo-3,4-dihydro-quinazolin-2-yl}-phenyl ester (101)

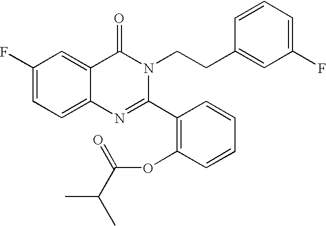

To a mixture of 6-fluoro-3-[2-(3-fluoro-phenyl)-ethyl]-2-(2-hydroxy-phenyl)-3H-quinazolin-4-one (0.3 g, 0.001 mol) in pyridine (5 mL), isobutyryl chloride (0.21 g, 0.002 mol) was added dropwise. The mixture was stirred at room temperature for 24 h and poured onto ice water. The precipitate was filtered off, washed with water, dried and recrystallized from ethanol to give 0.35 g (78%) of (101) as colorless crystals. GC/EI-MS, m/z (rel. int.) 448 (M⁺, 1), 377 (5), 257 (17), 256 (100), 137(11), 71 (7), 43 (16).

In a similar manner, the following compounds were made:

### 2,2-Dimethyl-propionic acid 2-{6-fluoro-3-[2-(3-fluoro-phenyl)-ethyl]4-oxo-3,4-dihydro-quinazolin-2-yl}-phenyl ester (102)

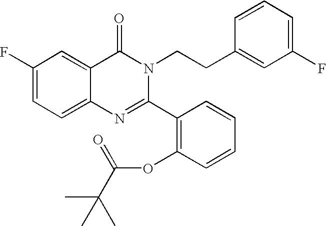

Prepared from 6-fluoro-3-[2-(3-fluoro-phenyl)-ethyl]-2-(2-hydroxy-phenyl)-3H-quinazolin-4-one (0.3 g, 0.001 mol) and tetramethylacetyl chloride (0.24 g, 0.002 mol) yielding 0.37 g (80%) of (102) as colorless crystals from ethanol. GC/EI-MS, m/z (rel. int.) 462 (M⁺, 1), 377 (7), 340 (18), 283 (21), 257 (17), 256 (100), 137 (17), 57 (54).

### Dimethyl-carbamic acid 2-{6-fluoro-3-[2-(3-fluoro-phenyl)-ethyl]-4-oxo-3,4-dihydro-quinazolin-2-yl}-phenyl ester (103)

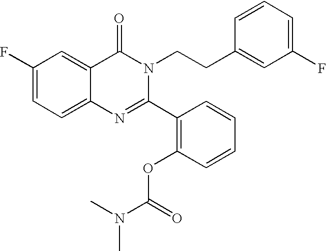

Prepared from 6-fluoro-3-[2-(3-fluoro-phenyl)-ethyl]-2-(2-hydroxy-phenyl)-3H-quinazolin-4-one (0.407 g, 0.0011 mol) and dimethyl carbamyl chloride (0.11 mL, 0.0012 mol) on refluxing overnight. Yield 0.234 g (50%) of (103) as colorless crystals from ethanol. ¹H NMR (CDCl₃): δ 8.00 (1H, dd, J₁=5.5, J₂=2.7), 7.79-7.73 (1H, m), 7.55-7.41 (3H, m), 7.33-7.27 (1H, m), 7.18-7.11 (2H, m), 6.90-6.84 (1H, m), 6.65 (1H, d, J=7.7), 6.58 (1H, dd, J₁=5.9, J₂=2.1), 4.46-4.37 (1H, m), 3.91-3.81 (1H, m), 2.94-2.80 (1H, m), 2.85 (3H, s), 2.74 (3H, s). ¹³C NMR (CDCl₃): δ 164.46, 162.76, 161.27, 159.46, 153.30, 152.12, 148.53, 143.88, 140.07, 139.97, 131.18, 130.09, 130.01, 128.92, 127.37, 125.32, 124.48, 123.28, 123.13, 122.96, 122.04, 121.93, 115.88, 115.61, 113.74, 113.47, 111.69, 111.37, 47.23, 36.67, 36.18, 34.03.

### EXAMPLE 10

### Preparation of methylamino-acetic acid 2-{6-fluoro-3-[2-(3-fluoro-phenyl)-ethyl]-4-oxo-3,4-dihydro-quinazolin-2-yl}-phenyl ester hydrochloride (105)

### (tert-Butoxycarbonyl-methyl-amino)-acetic acid 2-{6-fluoro-3-[2-(3-fluoro-phenyl)-ethyl]-4-oxo-3,4-dihydro-quinazolin-2-yl}-phenyl ester (104)

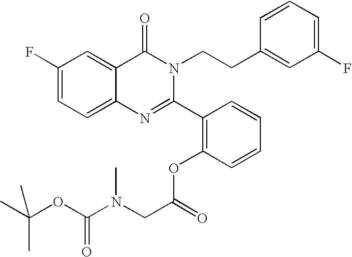

A mixture of 6-fluoro-3-[2-(3-fluoro-phenyl)-ethyl]-2-(2-hydroxy-phenyl)-3H-quinazolin-4-one (0.976 g, 0.0026 mol) and Boc-sarcosine (0.488 g, 0.0026 mol) in pyridine (2.1 mL) and ethylacetate (7.4 mL) was stirred at room temperature until the clear solution was formed. The mixture was cooled to −5° C., and 1,3-dicyclobexylcabodiimide (0.601 g, 0.0029 mol) in ethylacetate (3 mL) and pyridine (0.5 mL) was added. The mixture was allowed to warm to room temperature and stirred overnight. The precipitate (DCU) was filtered off, and the filtrate was washed with 5% sodium bicarbonate, 5% citric acid and water. The final organic layer was dried over MgSO₄ and the solvent was removed in vacuo. After purification by flash chromatography (1% MeOH/CHCl₃, R_{f=}0.45), 0.587 g (41%) of (104) was isolated as colorless crystals. ¹H NMR (DMSO-d₆): δ 7.90 (1H, d, J=7.9), 7.79-7.66 (3H, m), 7.57-7.40 (3H, m), 7.29-721 (1H, m), 7.05-6.99 (1H, m), 6.68-6.60 (2H, m), 4.28-4.18 (1H, m), 4.04 (2H, d, J=15.4), 3.77-2.64 (1H, m), 2.89-2.76 (2H, m), 2.62 (3H, d, J=3.8), 1.23 and 1.34 (9H, two s).

### Methylamino-acetic acid 2-{6-fluoro-3-[2-(3-fluoro-phenyl)-ethyl]-4-oxo-3,4-dihydro-quinazolin-2-yl}-phenyl ester hydrochloride (105)

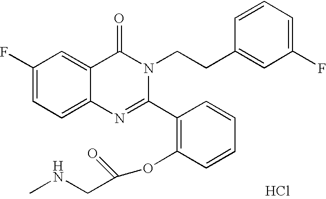

A mixture of (tert-butoxycarbonyl-methyl-amino)-acetic acid 2-{6-fluoro-3-[2-(3-fluoro-phenyl)-ethyl]-4-oxo-3,4-dihydro-quinazolin-2-yl}-phenyl ester (104) (0.283 g, 0.0051 mol) and trifluoroacetic acid (1.2 mL) was stirred at room temperature under nitrogen for 20 min. The solvent was removed in vacuo, the residue was dissolved in ethyl acetate (0.6 mL), and ethyl acetate saturated with HCl (1.5 mL) was added followed by addition of diethyl ether (2 mL). The mixture was refrigerated for 1 h, and the colorless crystals were filtered off, washed with diethyl ether and dried in vacuuo to give 0.189 g (76%) of (105). ¹H NMR (DMSO-d₆): δ 9.5 (2H, broad s), 7.92 (1H, d, J=7.8), 7.80-7.69 (3H, m), 7.58-7.48 (3H, m), 7.30-722 (1H, m), 7.06-6.98 (1H, m), 6.67-6.61 (2H, m), 4.28-4.18 (1H, m), 4.04 (2H, d, J=15.4), 3.75-2.63 (1H, m), 2.92-2.80 (2H, m), 2.62 (3H, m). ¹³C NMR (DMSO-d₆): δ 164.80, 163.28, 161.57, 160.04, 159.86, 159.81, 158.31, 158.0, 157.49, 150.60, 145.88, 143.09, 143.11, 140.15, 140.05, 130.92, 130.06, 129.95, 129.86, 129.75, 129.62, 126.81, 126.21, 123.93, 123.96, 122.75, 122.43, 121.61, 121.49, 114.68, 114.41, 113.08, 112.81, 110.48, 110.16, 47.20, 46.27, 32.58, 31.74.

### EXAMPLE 11

### Preparation of sodium salt of 6-fluoro-3-[2-(3-fluoro-phenyl)-ethyl]-2-(2-hydroxy-phenyl)-3H-quinazolin-4-one (106)

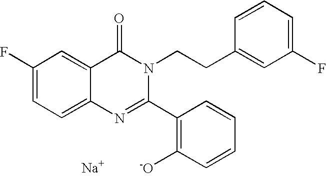

A solution of 6-fluoro-3-[2-(3-fluoro-phenyl)-ethyl]-2-(2-hydroxy-phenyl)-3H-quinazolin-4-one (43) (0.756 g, 0.002 mol) in dry acetonitrile (100 mL) was added dropwise under nitrogen to a suspension of sodium hydride (0.048 g, 0.002 mol) in dry acetonitrile (50 mL). The mixture was stirred under nitrogen overnight at room temperature. The light yellow precipitate was filtered off, washed with dry acetonitrile and dried in vacuo to give 0.65 g (81%) of (106) as light yellow powder. ¹H NMR (DMSO-d₆): δ 7.81-7.77 (1H, m), 7.67-7.64 (2H, m), 7.24-7.17 (1H, m), 6.99-6.92 (3H, m), 6.67 (1H, d, J=7.4), 6.58-6.53 (1H, m), 6.27 (1H, d, J=8.0), 6.07 (1H, t, J=7.0), 4.41-4.28 (2H, broad m), 2.87-2.62 (2H, broad m).

### EXAMPLE 12

### Preparation of phosphoric acid mono-(2-{6-fluoro-3-[2-(3-fluoro-phenyl)-ethyl]-4-oxo-3,4-dihydro-quinazolin-2-yl}-phenyl) ester (108)

### Phosphoric acid dibenzyl ester 2-{6-fluoro-3-[2-(3-fluoro-phenyl)-ethyl]-4-oxo-3,4-dihydro-quinazolin-2-yl}-phenyl ester (107)

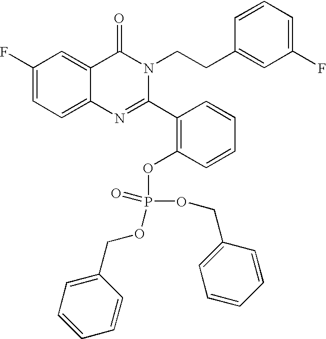

A stirred solution of 6-fluoro-3-[2-(3-fluoro-phenyl)-ethyl]-2-(2-hydroxy-phenyl)-3H-quinazolin-4-one (43) (0.95 g, 0.0025 mol) in dry acetonitrile (120 mL) was cooled under nitrogen to −10° C., and CCl₄ (1.9 g, 0.0125 mol) was added followed by N,N-dimethylaminopyridine (0.31 g, 0.0025 mol). One minute later, the addition of dibenzyl phosphite (0.94 g, 0.0036 mol) via syringe was begun. During this addition, the temperature in the flask was kept at −10° C. In 30 min after addition, 0.5 M aqueous KH₂PO₄ (90 mL) and acetonitrile (100 mL) were added, and the mixture was warmed to room temperature and kept overnight. The mixture was extracted with ethyl acetate (3×100 mL), the combined extracts were washed with water and brine. The organic layer was dried over anhydrous Na₂SO₄, and the solvent was removed in vacuo to give 1.34 g (85%) of pure (107) as colorless caramel. ¹H NMR (DMSO-d₆): δ 7.88-7.73 (1H, m), 7.76-7.72 (2H, m), 7.68-7.62 (1H, m), 7.51-7.47 (2H, m), 7.42-7.19 (9H, m), 7.14-6.99 (4H, m), 6.66-6.60 (2H, m), 5.06-4.86 (4H, m), 4.32-4.22 (1H, m), 3.70-3.60 (1H, m), 2.94-2.74 (1H, m).

### Phosphoric acid mono-(2-{6-fluoro-3-[2-(3-fluoro-phenyl)-ethyl]-4-oxo-3,4-dihydro-quinazolin-2-yl}-phenyl) ester (108)

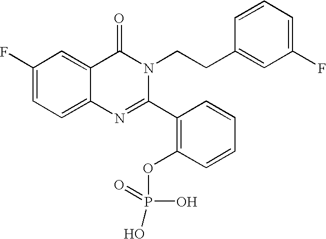

A mixture of phosphoric acid dibenzyl ester 2-{6-fluoro-3-[2-(3-fluoro-phenyl)-ethyl]-4-oxo-3,4-dihydro-quinazolin-2-yl}-phenyl ester (107) (1.34 g, 0.0021 mol), Pd/C (1.5 g), tetrahydrofuran (50 mL) and water (50 mL) was placed in atmosphere of hydrogen at normal pressure and kept at room temperature overnight. The mixture was filtered off, and the residue was washed with water (50 mL). The combined filtrates were placed on rotavapor, and tetrahydrofuran and water were distilled off until the final residual volume reached about 10 mL. The precipitate formed was filtered off and dried in vacuo to give 0.48 g (50%) of pure (108) as colorless powder. ¹H NMR (DMSO-d₆): δ 7.91 (1H, d, J=8.2), 7.79-7.77 (2H, m), 7.58-7.53 (2H, m), 7.34 (1H, d, J=7.1), 7.27-7.20 (2H, m), 7.04-6.97 (1H, m), 6.68-6.61 (2H, m), 4.37-4.27 (1H, m), 3.75-3.65 (1H, m), 2.97-2.88 (1H, m), 2.83-2.50 (1H, m).

### EXAMPLE 13

### Preparation of 7-fluoro-3-[2-(3-fluoro-phenyl)-ethyl]-2-(2-hydroxy-phenyl)-3H-quinazolin-4-one (109)

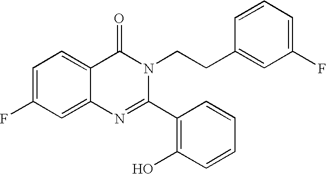

An attempt to synthesize compound 109 by conventional Method A as described for compound 17 resulted in the corresponding 7-amino-substituted compound 76. The use of microwave assisted conditions as described in Method C for preparing compound 17 gave the desired compound 109. A dried heavy-walled Pyrex tube was charged with acetic acid 2-(7-fluoro-4-oxo-4H-benzo[d][1,3]oxazin-2-yl)-phenyl ester (0.28 g, 0.001 mol) and 2-(3-fluoro-phenyl)-ethylamine (0.242 g, 0.002 mol) in DMF (1 mL). The screw cap was tightened thoroughly. The reaction mixture was exposed to microwave irradiation at 240° C. for 10 min. The reaction tube was allowed to reach room temperature, and the reaction mixture was poured into a mixture of ice (10 g) and 10% aqueous HCl (10 mL) and vigorously stirred. Diethyl ether was added (5 mL), and the mixture was vigorously stirred again. The upper ethereal layer was allowed to evaporate at room temperature, and the solid product was separated, washed with water (5 mL) and recrystallized from ethanol to give 0.31 g (99%) of colorless crystals. GC/EI-MS, m/z (rel. int.) 378 (M⁺, 3).

In order to use a compound of Formula (I) or a pharmaceutically acceptable salt thereof for the treatment of humans and other mammals, it is normally formulated in accordance with standard pharmaceutical practice as a pharmaceutical composition.

The calcilytic compounds can be administered by different routes including intravenous, intraperitoneal, subcutaneous, intramuscular, oral, topical (transdermal), or transmucosal administration. For systemic administration, oral administration is preferred. For oral administration, for example, the compounds can be formulated into conventional oral dosage forms such as capsules, tablets, and liquid preparations such as syrups, elixirs, and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various calcilytic compounds to be administered can be determined by standard procedures taking into account factors such as the compound IC₅₀, EC₅₀, the biological half-life of the compound, the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula (I). A topical formulation contains suitably 0.01 to 5.0% of a compound of Formula (I). The active ingredient may be administered, for example, from 1 to 6 times per day, preferably once, sufficient to exhibit the desired activity, as is readily apparent to one skilled in the art.

As used herein, “treatment” of a disease includes, but is not limited to prevention, retardation and prophylaxis of the disease.

Diseases and disorders which might be treated or prevented, based upon the affected cells, include bone and mineral-related diseases or disorders; hypoparathyroidism; those of the central nervous system such as seizures, stroke, head trauma, spinal cord injury, hypoxia-induced nerve cell damage, such as occurs in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, and Tourette's syndrome; diseases involving excess water reabsorption by the kidney, such as syndrome of inappropriate ADH secretion (SIADH), cirrhosis, congestive heart failure, and nephrosis; hypertension; preventing and/or decreasing renal toxicity from cationic antibiotics (e.g., aminoglycoside antibiotics); gut motility disorders such as diarrhea and spastic colon; GI ulcer diseases; GI diseases with excessive calcium absorption such as sarcoidosis; autoimmune diseases and organ transplant rejection; squamous cell carcinoma; and pancreatitis.

In a preferred embodiment of the present invention, the present compounds are used to increase serum parathyroid hormone (“PTH”) levels. Increasing serum PTH levels can be helpful in treating diseases such as hypoparathyroidism, osteosarcoma, periodontal disease, fracture, osteoarthritis, rheumatoid arthritis, Paget's disease, humoral hypercalcemia malignancy and osteoporosis.

In a preferred embodiment of the present invention, the present compounds are co-administered with an anti-resorptive agent. Such agents include, but are not limited estrogen, 1, 25 (OH)₂ vitamin D3, calcitonin, selective estrogen receptor modulators, vitronectin receptor antagonists, V-H+-ATPase inhibitors, src SH2 antagonists, bisphosphonates and cathepsin K inhibitors.

Another aspect of the present invention describes a method of treating a patient comprising administering to the patient an amount of a present compound sufficient to increase the serum PTH level. Preferably, the method is carried out by administering an amount of the compound effective to cause an increase in duration and/or quantity of serum PTH level sufficient to have a therapeutic effect.

In various embodiments, the compound administered to a patient causes an increase in serum PTH having a duration of up to one hour, about one to about twenty-four hours, about one to about twelve hours, about one to about six hours, about one to about five hours, about one to about four hours, about two to about five hours, about two to about four hours, or about three to about six hours.

In an alternative embodiment of the present invention, the compound administered to a patient causes an increase in serum PTH having a duration of more than about twenty four hours provided that it is co-administered with an anti resorptive agent.

In additional different embodiments, the compound administered to a patient causes an increase in serum PTH of up to two fold, two to five fold, five to ten fold, and at least 10 fold, greater than peak serum PTH in the patient. The peak serum level is measured with respect to a patient not undergoing treatment.

Composition of Formula (I) and their pharmaceutically acceptable salts, which are active when given orally, can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

No unacceptable toxological effects are expected when compounds of the present invention are administered in accordance with the present invention.

The biological activity of the compounds of Formula (I) are demonstrated by the following tests:

(I) Calcium Receptor Inhibitor Assay

Calcilytic activity was measured by determining the IC₅₀ of the test compound for blocking increases of intracellular Ca²⁺ elicited by extracellular Ca²⁺ in HEK 293 4.0-7 cells stably expressing the human calcium receptor. HEK 293 4.0-7 cells were constructed as described by Rogers et al., J. Bone Miner. Res. 10 (Suppl. 1):S483, 1995 (hereby incorporated by reference herein). Intracellular Ca²⁺ increases were elicited by increasing extracellular Ca²⁺ from 1.0 to 1.3 mM. Intracellular Ca²⁺ was measured using fluo-3, a fluorescent calcium indicator (Biotium).

The procedure was as follows:
1. Cells were maintained in DMEM with 10% FBS and 200 μg/ml hygromycin, under 5% CO₂ at 37° C.
2. At 24-hours prior to analysis, the cells were trypsinized and plated in the above medium at 120,000 cells/well in black sided, clear-bottom, collagen I coated, 96-well plates. Plates were centrifuged at 800 rpm for 2 minutes and incubated under 5% CO₂ at 37° C. overnight.
3. The media was then aspirated and 80 μL/well of 6 μM fluo-3 in assay buffer was added to the plate. Assay buffer contains 20 mM Na-Hepes, pH 7.4, 126 mM NaCl, 5 mM KCl, 1 mM MgCl₂, 1 mM CaCl₂, 1 mg/mL D-glucose and 1 mg/mL of bovine serum albumin (BSA; fraction V, ICN).
4. Cell-plates containing the fluo-3 solution were incubated in the dark, at room temperature, for 60 minutes. Following incubation plates were washed once, then refilled with 160 μL/well of assay buffer.
5. Measurements of fluorescence were performed using the FLIPR system (Molecular Devices), with a laser setting of 0.8 W and a 0.4 second CCD camera shutter speed. A two-addition protocol was used with a 40 μL addition of buffer or test compound 95 seconds before the addition of extracellular Ca²⁺. The extracellular [Ca²⁺] is increased from 1.0 to 1.3 mM by adding 50 μL of 2.5 mM CaCl₂ in assay buffer.
6. Calcilytic activity was determined by a compound's ability to block, in a concentration-dependent manner, increases in the concentration of intracellular Ca²⁺ elicited by increases in extracellular Ca²⁺. Fluorescence signals were measured as the peak height of the response and normalized to the response elicited by extracellular Ca²⁺ in the absence of test compound. All compounds were tested at 8 concentrations in duplicate with the highest concentration being 30 μM.

In general, those compounds having lower IC₅₀ values in the Calcium Receptor Inhibitor Assay are more preferred compounds. Compounds having an IC₅₀ greater than 30 uM were considered to be inactive. Preferred compounds are those having an IC₅₀ of 10 uM or lower, more preferred compounds have an IC₅₀ of 1 uM, and most preferred compounds have an IC₅₀ of 0.2 uM or lower.

(II) Calcium Receptor Binding Assay

HEK 293 4.0-7 cells stably transfected with the Human Parathyroid Calcium Receptor (“HuPCaR”) were scaled up in T180 tissue culture flasks. Plasma membrane is obtained by polytron homogenization or glass douncing in buffer (50 mM Tris-HCl, pH 7.4, 1 mM EDTA, 3 mM MgCl₂) in the presence of a protease inhibitor cocktail containing 1 μM Leupeptin, 0.04 μM Pepstatin, and 1 mM PMSF. Aliquoted membrane was snap frozen and stored at −80° C. The radioligand was radiolabeled with tritium to a radiospecific activity of 44 Ci/mmole and was aliquoted and stored in liquid nitrogen for radiochemical stability.

A typical reaction mixture contains 2 nM ³H compound ((R,R)-N-4′-Methoxy-t-3-3′-methyl-1′-ethylphenyl-1-(1-naphthyl)ethylamine), or ³H compound (R)-N-[2-hydroxy-3-(3-chloro-2-cyanophenoxy)propyl]-1,1-dimethyl-2-(4-methoxyphenyl)ethylamine, and 4-10 μg membrane in homogenization buffer containing 0.1% gelatin and 10% ethanol, in a reaction volume of 0.5 mL. Incubation is performed in 12×75 polyethylene tubes in an ice water bath. To each tube 25 μL of test sample in 100% ethanol is added, followed by 400 μL of cold incubation buffer, and 25 μL of 40 nM ³H-compound in 100% ethanol for a final concentration of 2 nM. The binding reaction is initiated by the addition of 50 μL of 80-200 μg/mL HEK 293 4.0-7 membrane diluted in incubation buffer, and allowed to incubate at 4° C. for 30 min. Wash buffer is 50 mM Tris-HCl containing 0.1% PEI. Nonspecific binding is determined by the addition of 100-fold excess of unlabeled homologous ligand, and is generally 20% of total binding. The binding reaction is terminated by rapid filtration onto 1% PEI pretreated GF/C filters using a Brandel Harvestor. Filters are placed in scintillation fluid and radioactivity assessed by liquid scintillation counting. All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the area can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A compound having the chemical formula: 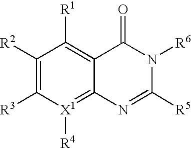
wherein:
R¹, R² and R³ is each independently selected from one of: H, halogen, CN, CF₃, OCF₃, lower alkyl, lower alkoxy, NH-acetyl, NH-lower alky, NH-alkylaryl; N(lower alkyl)₂, C(O)OH, C(O)O-lower alkyl, C(O)NH-lower alkyl, C(O)N(lower alkyl)₂, OH, OC(O)-lower alkyl, OC(O)-lower alkyalamino, OC(O)-lower alkyl-N(lower alkyl)₂, and OP(O)(OH)₂;
R⁴ is optional and may be selected from one of: H, halogen, CN, CF₃, OCF₃, lower alkyl, lower alkoxy, NH-acetyl, NH-lower alky, NH-alkylaryl, N(lower alkyl)₂, C(O)OH, C(O)O-lower alkyl, C(O)NH-lower alkyl, C(O)N(lower alkyl)₂, OH, OC(O)-lower alkyl, OC(O)-lower alkyalamino, OC(O)-lower alkyl-N(lower alkyl)₂, and OP(O)(OH)₂;
X¹ is selected from one of C and N,
R⁵ is selected from one of: H lower alkyl, a furyl, thienyl, styryl, pyridyl and phenyl group optionally substituted with 1 to 3 substituents selected from one of H, halogen, CN, CF₃, OCF₃, lower alkyl, NH-alkylaryl, N(lower alkyl)₂, lower alkoxy, OH, OC(O)-lower alkyl, OC(O)-lower alkyalamino, OC(O)-lower alkyl-N(lower alkyl)₂, and OP(O)(OH)₂;
R⁶ is selected from one of: H, lower alkyl, and a group comprising —(CH₂)ₙ—X²—R⁷ wherein n is 0, 1, or 2, X² is O, C(O), CH(OH), lower alkyl or a single bond, and
R⁷ is an aromatic group optionally substituted with 1 to 3 substituents selected from one of: H, halogen, CN, CF₃, OCF₃, lower alkyl, lower alkoxy, NH-lower alkyl, NH-alkylaryl, N(lower alkyl)₂, OH, OC(O)-lower alkyl, OC(O)-lower alkyalamino, OC(O)-lower alkyl-N(lower alkyl)₂, and OP(O)(OH)₂;
or a pharmaceutically acceptable salt or complex thereof.

2. A compound according to claim 1, wherein R¹, R², R³, and R⁴ are independently selected from one of hydrogen, halogen, lower alkyl, OH and OP(O)(OH)₂.

3. A compound according to claim 2, wherein said halogen is selected from one of fluorine and chlorine.

4. A compound according to claim 2, wherein lower alkyl is methyl.

5. A compound according to claim 2 wherein, R¹ is selected from one of hydrogen and methyl.

6. A compound according to claim 2, wherein R² is selected from one of hydrogen, fluorine, chlorine, hydroxy, and methyl.

7. A compound according to claim 2, wherein R³ is selected from one of hydrogen and chlorine.

8. A compound according to claim 2, wherein R⁴ is selected from one of hydrogen, hydroxy, and methyl.

9. A compound according to claim 1, wherein X¹ is carbon.

10. A compound according to claim 1, wherein R⁵ is phenyl optionally substituted with 1 or 2 hydroxy.

11. A compound according to claim 1, wherein R⁶ further comprises the group —(CH₂)ₙ—X²—R⁷;
wherein n is 1 or 2;
X² is a single bond, and
R⁷ is phenyl optionally substituted with 1 or 2 halogens.

12. A compound according to claim 11, wherein n is 2 and said halogens are selected from one of fluorine and chlorine.

13. A pharmaceutical composition comprising a compound according to claim 1 and pharmaceutically acceptable diluent or excipient.

14. A method of treating disease or disorder characterized by abnormal bone or mineral homeostasis which comprises the administration to a subject in need of treatment thereof an effective amount of a compound according to claim 1.

15. A method according to claim 14, wherein the bone or mineral disorder is selected from one of osteosarcoma, periodontal disease, fracture healing, osteoathritis, rheumatoid arthritis, Paget's dicease, humoral hypercalcemia malignancy, and osteoporosis.

16. A method according to claim 14, wherein the bone or mineral disease or disorder is osteoporosis.

17. A method of increasing serum parathyroid hormone levels in mammals, which comprises the administration to a subject in need of treatment thereof an effective amount of a compound according to claim 1.

18. A method for preparing 2,3,5,6,7,8-substituted 3H-quinazolin-4-ones by reacting 2,4,5,6,7,8-substituted benzo[d][1,3]oxazin-4-ones with primary amines under microwave irradiation conditions.

19. The compound according to claim 1, wherein compound is selected from one of:
2-(2-hydroxy-phenyl)-3-phenethyl-3H-quinazolin-4-one;
2-(2,5-dihydroxy-phenyl)-3-phenethyl-3H-quinazolin-4-one;
2-(3-hydroxy-phenyl)-3-phenethyl-3H-quinazolin-4-one;
2-(2-hydroxy-phenyl)-3-(2-phenoxy-ethyl)-3H-quinazolin-4-one;
3-[2-(4-fluoro-phenyl)-ethyl]-2-(2-hydroxy-phenyl)-3H-quinazolin-4-one;
3-[2-(3-fluoro-phenyl)-ethyl]-2-(2-hydroxy-phenyl)-3H-quinazolin-4-one;
3-[2-(2-fluoro-phenyl)-ethyl]-2-(2-hydroxy-phenyl)-3H-quinazolin-4-one;
3-[2-(3-chloro-phenyl)-ethyl]-2-(2-hydroxy-phenyl)-3H-quinazolin-4-one;
3-[2-(2-chloro-phenyl)-ethyl]-2-(2-hydroxy-phenyl)-3H-quinazolin-4-one;
2-(2-hydroxy-phenyl)-3-[2-(4-methoxy-phenyl)-ethyl]-3H-quinazolin-4-one;
2-(2-hydroxy-phenyl)-3-(2-p-tolyl-ethyl)-3H-quinazolin-4-one;
2-(2-hydroxy-phenyl)-6-methyl-3-phenethyl-3H-quinazolin-4-one;
6-fluoro-2-(2-hydroxy-phenyl)-3-phenethyl-3H-quinazolin-4-one;
6-chloro-2-(2-hydroxy-phenyl)-3-phenethyl-3H-quinazolin-4-one;
2-(2-hydroxy-phenyl)-3-phenethyl-5-phenethylamino-3H-quinazolin-4-one;
2-(2-hydroxy-phenyl)-5-methyl-3-phenethyl-3H-quinazolin-4-one;
7-chloro-2-(2-hydroxy-phenyl)-3-phenethyl-3H-quinazolin-4-one;
2-(2-hydroxy-phenyl)-8-methyl-3-phenethyl-3H-quinazolin-4-one;
6-fluoro-3-[2-(3-fluoro-phenyl)-ethyl]-2-(2-hydroxy-phenyl)-3H-quinazolin-4-one;
6-fluoro-3-[2-(2-fluoro-phenyl)-ethyl]-2-(2-hydroxy-phenyl)-3H-quinazolin-4-one;
7-fluoro-3-[2-(3-fluoro-phenyl)-ethyl]-2-(2-hydroxy-phenyl)-3H-quinazolin-4-one;
3-[2-(2-fluoro-phenyl)-ethyl]-2-(2-hydroxy-phenyl)-5-methyl-3H-quinazolin-4-one;
3-[2-(3-fluoro-phenyl)-ethyl]-2-(2-hydroxy-phenyl)-5-methyl-3H-quinazolin-4-one;
3-[2-(3-fluoro-phenyl)-ethyl]-2-(2-hydroxy-phenyl)-6-methyl-3H-quinazolin-4-one;
3-[2-(2-fluoro-phenyl)-ethyl]-2-(2-hydroxy-phenyl)-6-methyl-3H-quinazolin-4-one;
6-chloro-3-[2-(3-fluoro-phenyl)-ethyl]-2-(2-hydroxy-phenyl)-3H-quinazolin-4-one;
6-chloro-3-[2-(2-fluoro-phenyl)-ethyl]-2-(2-hydroxy-phenyl)-3H-quinazolin-4-one;
3-[2-(3-fluoro-phenyl)-ethyl]-2-(2-hydroxy-phenyl)-6-methoxy-3H-quinazolin-4-one;
3-[2-(3-fluoro-phenyl)-ethyl]-6-hydroxy-2-(2-hydroxy-phenyl)-3H-quinazolin-4-one; acetic acid 2-{6-fluoro-3-[2-(3-fluoro-phenyl)-ethyl]-4-oxo-3,4-dihydro-quinazolin-2-yl}-phenyl ester;
3-[2-(3-fluoro-phenyl)-ethyl]-2-(2-hydroxy-phenyl)-8-methoxy-3H-quinazolin-4-one, isobutyric acid 2-{6-fluoro-3-[2-(3-fluoro-phenyl)-ethyl]-4-oxo-3,4-dihydro-quinazolin-2-yl}-phenyl ester;
sodium salt of 6-fluoro-3-[2-(3-fluoro-phenyl)-ethyl]-2-(2-hydroxy-phenyl)-3H-quinazolin-4-one;
8-chloro-2-(2-hydroxy-phenyl)-3-phenethyl-3H-quinazolin-4-one;
7-chloro-3-[2-(3-fluoro-phenyl)-ethyl]-2-(2-hydroxy-phenyl)-3H-quinazolin-4-one;
7-chloro-3-[2-(2-fluoro-phenyl)-ethyl]-2-(2-hydroxy-phenyl)-3H-quinazolin-4-one;
2-(2-hydroxy-phenyl)-3-(2-pyridin-3-yl-ethyl)-3H-quinazolin-4-one;
6-fluoro-2-(2-hydroxy-phenyl)-3-(2-pyridin-3-yl-ethyl)-3H-quinazolin-4-one;
2-(2-hydroxy-phenyl)-3-phenethyl-3H-pyrido[2,3-d]pyrimidin-4-one;
3-[2-(3-fluoro-phenyl)-ethyl]-2-(2-hydroxy-phenyl)-3H-pyrido[2,3-d]pyrimidin-4-one;
3-(1,1-dimethyl-3-phenyl-propyl)-6-fluoro-2-(2-hydroxy-phenyl)-3H-quinazolin-4-one;
methylamino-acetic acid 2-{6-fluoro-3-[2-(3-fluoro-phenyl)-ethyl]-4-oxo-3,4-dihydro-quinazolin-2-yl}-phenyl ester hydrochloride;
6-fluoro-2-(2-hydroxy-phenyl)-3-(2-phenyl-propyl)-3H-quinazolin-4-one;
6-fluoro-2-(2-hydroxy-phenyl)-3-(R-2-phenyl-propyl)-3H-quinazolin-4-one;
6-fluoro-2-(2-hydroxy-phenyl)-3-(S-2-phenyl-propyl)-3H-quinazolin-4-one; and
6-fluoro-2-(2-hydroxy-phenyl)-3-(3-phenyl-propyl)-3H-quinazolin-4-one.
